(19) United States
(12) Patent Application Publication
(10) Pub. No.: US 2025/0135196 A1
(43) Pub. Date: May 1, 2025

(54) COMPOSITIONS, SYSTEMS, AND METHODS FOR TREATING CANCER USING TUMOR TREATING FIELDS WITH INHIBITORS OF MIF, MICA, AND/OR MICB

(71) Applicants: Brown University, Providence, RI (US); Novocure GmbH, Root D4 (CH)

(72) Inventors: Wafik El-Deiry, Barrington, RI (US); Praveen Srinivasan, Providence, RI (US); Tali Voloshin-Sela, Haifa (IL)

(21) Appl. No.: 18/927,782

(22) Filed: Oct. 25, 2024

Related U.S. Application Data
(60) Provisional application No. 63/601,599, filed on Nov. 21, 2023.
(60) Provisional application No. 63/593,790, filed on Oct. 27, 2023.

(51) Int. Cl. A61N 1/36; A61K 45/06; A61N 1/40; A61P 35/00; C07K 16/28; G16H 20/17

(52) U.S. Cl. CPC A61N 1/36002; A61K 45/06; A61N 1/40; A61P 35/00; C07K 16/2833; G16H 20/17

(57) ABSTRACT

Compositions, systems, and advanced methods for reducing viability of cancer cells and treating cancer, as well as preventing an increase of volume of a tumor present in a body of a living subject, are disclosed. The systems and methods involve application of an alternating field concurrently with administration of at least one composition that inhibits at least one of immunomodulatory cytokine such as (but not limited to) macrophage migration inhibitory factor (MIF), MHC class I chain-related polypeptide A (MICA), and/or MHC class I chain-related polypeptide B (MICB).

### CROSS-REFERENCE TO RELATED APPLICATIONS

This application claims the benefit of priority to U.S. Provisional Patent No. 63/593,790, filed on 27 Oct. 2023, the entirety of which is incorporated by reference as if fully reproduced and set forth herein in its entirety. This application also claims the benefit of priority to U.S. Provisional Patent No. 63/601,599, filed on 21 Nov. 2023, the entirety of which is incorporated by reference as if fully reproduced and set forth herein in its entirety.

### STATEMENT REGARDING FEDERALLY SPONSORED RESEARCH OR DEVELOPMENT

Not Applicable.

### FIELD OF THE INVENTION

The present invention relates to synergistic and lifesaving effects with Tumor treating fields (TTFields), which is an FDA-approved therapy for the treatment of glioblastoma and malignant pleural mesothelioma, in particular, to enhanced effects and potentially lifesaving advantages that arise when TTFields are used concurrently with administration of at least one composition that inhibits at least one immunomodulatory cytokine optionally with measurements of effects on cancer cells (e.g., RNA-seq, transcriptome and/or secretome effects on cancer cells).

### BACKGROUND OF THE INVENTION

Tumor Treating Fields (TTFields) are low intensity (e.g., 1-3 V/cm) alternating electric fields within the intermediate frequency range (such as, but not limited to, 100-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the electrode arrays that make up each of these pairs are positioned on opposite sides of the body part that is being treated. More specifically, for the OPTUNE® system, one pair of electrodes is located to the left and right (LR) of the tumor, and the other pair of electrodes is located anterior and posterior (AP) to the tumor. TTFields are approved for the treatment of glioblastoma multiforme (GBM), and may be delivered, for example, via the OPTUNE® system (e.g., Novocure Limited, St. Helier, Jersey), which includes transducer arrays placed on the patient's shaved head.

Each transducer array used for the delivery of TTFields in the OPTUNE® device comprises a set of ceramic disk electrodes, which are coupled to the patient's skin (such as, but not limited to, the patient's shaved head for treatment of GBM) through a layer of conductive medical gel. The purpose of the medical gel is to deform to match the body's contours and to provide good electrical contact between the arrays and the skin; as such, the gel interface bridges the skin and reduces interference. The device is intended to be continuously worn by the patient for 2-4 days before removal for hygienic care and re-shaving (if necessary), followed by reapplication with a new set of arrays. As such, the medical gel remains in substantially continuous contact with an area of the patient's skin for a period of 2-4 days at a time. In addition, the arrays can be shifted a few centimeters in either direction to allow the skin to heal from one period of treatment to the next. Therefore, a portion of skin that was covered by electrodes/gel for a 2-4 day period could then be uncovered for 2-4 days when the replaced electrodes are shifted slightly; then the device may be reapplied to the original portion of skin for the next 2-4 day period.

Glioblastoma (“GBM” or glioblastoma multiforme), an aggressive and common malignant brain tumor, is on the rise globally (Grech, et al., 2020). Tumor treating fields (TTFields) is an FDA-approved therapy for GBM and malignant pleural mesothelioma. TTFields is being studied for treatment of other cancers such as non-small cell lung cancer. TTFields may have far reaching applications for cancer.

The anti-tumor effects of TTFields were first observed by Dr. Yoram Palti, founder of Novocure® (NASDAQ: NVCR), from the Technion University in Israel. By tuning the frequency of alternating electric fields to about 100-300 kHz, Dr. Palti observed dividing tumor cells underwent cell blebbing and cell death. TTFields was initially found to work by disrupting tumor cells as they undergo mitosis by interrupting proteins with large dipole moments that are critical for executing cytokinesis and segregation of sister chromatids. The technology worked with few side effects. Known side effects are primarily localized to the skin after long use (e.g., after about >18 hours per day or longer) of the ceramic application transducers in contact with the skin. One TTFields device is intended to be continuously worn by the patient for 2-4 days before removal for hygienic care and re-shaving of skin (if necessary), followed by reapplication with a new set of transducer arrays.

As such, the medical gel remains in substantially continuous contact with an area of the patient's skin for a period of 2-4 days at a time. In addition, the arrays can be shifted a few centimeters in either direction to allow the skin to heal from one period of treatment to the next. Therefore, a portion of skin that was covered by electrodes/gel for a 2-4-day period could then be uncovered for 2-4 days when the replaced electrodes are shifted slightly; then the device may be reapplied to the original portion of skin for the next 2-4 day period. While TTFields has undergone rigorous, various clinical trials, the adverse effects of TTFields in published trials to date have included topical skin rashes caused by prolonged electrode/transducer use, not only on the scalp but on other bodily treatment areas. Importantly, since the initial proof-of-concept study published in 2007, the use of TTFields has become integrated into the standard-of-care multi-modality treatment of GBM.

While TTFields were initially demonstrated to inhibit cancer cell proliferation by interfering with mitotic apparatus, later work supported that TTFields show a broad mechanism of action by disrupting a multitude of biological processes, including DNA repair, cell permeability and immunological responses (Rominiyi, et al., 2021).

The leading TTField-generating devices are manufactured by the same founding company Novocure®. These devices are approved in the United States and Europe for the treatment of newly diagnosed and recurrent GBM and are undergoing clinical trials for several other tumor types. A specific TTFields device, manufactured under the trade name Optune® (formerly NovoTTF-100A or currently NovoTTF-200A, see the Novocure® LUNAR 2023 in References), is approved in the United States, Canada, Japan, Israel, and multiple countries in Europe for the treatment of newly diagnosed and recurrent GBM. The devices can be used in conjunction with other patterns of care for patients and can be deployed in active lifestyle settings, but unfortunately are only available in certain treatment centers, and require specific training and certification on the part of the prescribing physician.

When a TTFields device is used on the head, electrode arraysare placed onto a patient's shaved scalp. When not in use, the device's batteries are plugged into a power outlet to be re-charged. Among cancers, GBM has a very bleak survival prognosis, and TTFields has shown promise in treating more than just GBM. TTFields have been studied clinically together with standard-of-care (SOC) immunotherapy or chemotherapy regimens for treatment of non-small cell lung cancer that has progressed on platinum-based therapy (Leal, et al. (2023) Lancet Oncol, 24(9):1002-1017) and preclinically together with immune checkpoint inhibitors (Voloshin, et al. (2020) Cancer Immunol Immunother, 69(7):1191-1204; Barsheshet, et al. (2022) Int J Mol Sci, 23(22):14073). It is known that incidence of glioblastoma is dramatically rising; what is urgently needed are new treatment options with TTFields to save lives. There is also an urgent need for methods to improve treatments including TTFields by discovering enhanced, synergistic advantages that arise when TTFields are used in combination therapies in treatments of cancers.

### BRIEF SUMMARY OF THE INVENTION

The following presents a summary of the innovation to introduce some aspects of the invention. This summary is not an extensive overview of the invention. It is intended to neither identify all key or critical elements of the invention nor limit the scope of the invention. Its purpose is to introduce some concepts of the invention as a prelude to the more detailed description presented later. By reading the Summary, studying FIGS. 1-4, reading the Detailed Description, studying the Examples and the Claims, the inventive concepts can be initially discerned.

In a summary level of an embodiment, the present invention relates to synergistic, enhanced and potentially lifesaving advantages that arise when TTFields are used with measurements of effects on cancer cells (e.g., transcriptome and/or secretome effects on cancer cells; RNA-seq); then the measurements are used to intelligently or flexibly select combination therapies (e.g., with selected therapeutic agents) in treatments of cancers; and highly enhanced, lifesaving treatments are thereby provided.

Disclosed herein are biomarkers and rationales for therapeutic combinations exploiting molecular alterations induced upon cancer cells by TTFields. The molecular alterations are found by, for example, measuring changes in a transcriptome and/or a secretome of the cancer cells during/after the TTFields are applied to said cells. The molecular alterations are then exploited to further the clinical treatment (e.g., further induce cytostatic and/or cytotoxic effects) on the cancer cells.

In some embodiments, the technology disclosed herein provides a method for reducing viability of cancers cells comprising the steps of: (1) applying an alternating electric field (TTFields) to the cancer cells, and (2) contacting the cancer cells with a therapeutic agent; whereby the efficacy of the TTFields is increased compared to the application of TTFields without contacting the cancer cells with the therapeutic agent.

In some embodiments, the technology provides a method for inducing cytostatic and/or cytotoxic effects on a cancer cell, the method comprising the steps of (e.g., FIG. 1): (1) applying an alternating electric field or tumor treating fields (TTFields) to the cancer cell; and (2) contacting the cancer cell with a therapeutic agent, before, after, and/or during execution of step (1). According to some aspects, the method can further comprise the step of: (1b) measuring a transcriptome and/or a secretome of the cell after or during execution of step (1).

In some embodiments, the method can further comprise the step of: (0b) measuring a transcriptome and/or a secretome of the cell before execution of step (1).

In the above described embodiments, the method can be wherein step (1b) is performed after execution of step (1) and one or more changes induced in the transcriptome and/or secretome of the cancer cell after the application of the TTFields is determined via comparing a data measured before execution of step (1) (i.e., a data 0b or a pre-treatment data) and a data measured after execution of step (1) (i.e., a data 1b or a post-treatment data).

It is contemplated herein (Examples 1-4) that the method can be configured as comprising steps of a method of treating a subject in need of a cancer treatment, such that that the following method steps are executed: (0) obtaining a subject in need of a cancer treatment; (0b) optionally measuring a transcriptome and/or a secretome of the cancer whereby a pre-treatment data is obtained; (1) applying an alternating electric field or tumor treating fields (TTFields) to the cancer; (1b) optionally measuring a transcriptome and/or a secretome of the cancer after or during execution of step (1), whereby a post-treatment data is obtained; and (2) contacting the cancer with a therapeutic agent, before, after, or during an execution of step (1); wherein the contacting is done by an administration of a therapeutic agent to the subject in need thereof, or an administration of a pharmaceutical formulation thereof.

In some embodiments, the methods described above can be wherein the pre-treatment data and/or the post-treatment data is/are compared and/or utilized to determine the therapeutic agent utilized in step (2) and/or a dose or a dose regime of the agent.

According to some aspects, the methods disclosed herein can be wherein the order of the execution of the steps is changed, the method is repeated or wherein the method is executed as a continuous method.

In some embodiments, the methods can be executed wherein the pre-treatment and/or post-treatment data is compared to a previously acquired data from a different subject or to a data contained in a database, wherein the comparison includes a machine learning, sending to another location, a comparison by a healthcare provider, and/or a storing for future access.

In the methods disclosed herein, the cytostatic and/or cytotoxic effects on the cancer cell can be greater than when compared to the same effects of applying TTFields (1) without contacting the cancer cell with the therapeutic agent (2) or vice versa; whereby the cytostatic and/or cytotoxic effects on the cancer cell can be greater than when compared to contacting the cancer cell with the therapeutic agent (2) without applying the TTFields (1).

In some embodiments, the cancer cell is a cell of a subject in need of a treatment thereof and the method as practiced further induces an anti-tumor immunity or an immune response against a cancer cell in the subject.

In some embodiments, the methods discussed above can be whereby execution of step (1) induces a change, upregulation, downregulation, or modulation in a transcriptome and/or a secretome of the cancer (cell) and whereby step (2) either upregulates or downregulates said change. According to some aspects, the therapeutic agent can comprise a modulator of macrophage migration inhibitory factor (MIF) immunomodulatory cytokine. In some embodiments, MIF is upregulated independently of the p53 pathway by an execution of step (1). The modulator, in some embodiments, comprises a small molecule inhibitor of MIF, an antibody inhibitor of MIF, an anti-MIF antibody, an MIF down-regulator, or an agent that targets MIF receptors. The method can be, according to some aspects, wherein the modulator comprises an anti-CD74 monoclonal antibody, Reparixin, ISO-1, 4-IPP, Ibudilast, RTL100, BAX69, or NbE-10 (Sparkes, et al., 2018; Kok, et al., 2018).

In some embodiments, the therapeutic agent comprises a modulator of Interleukin 8 (IL-8) and/or whereby IL-8 is downregulated by an execution of step (1). In this example, the modulator can comprise a small molecule inhibitor of IL-8, an antibody inhibitor of IL-8, or an IL-8 down-regulator. According to some aspects, IL-8 is downregulated independently of the p53 pathway.

In some embodiments, the therapeutic agent comprises a modulator of major histocompatibility complex (MHC) class I chain related-proteins A (MICA) and/or B (MICB) and whereby MICA and/or MICB are each upregulated by an execution of step (1). For example, MICA and/or MICB can each or separately be upregulated independently of the p53 pathway. In this example, the modulator can comprise a small molecule inhibitor of MICA and/or MICB and/or inhibitor of shedding of MICA and/or MICB, an antibody inhibitor of MICA and/or MICB and/or inhibitor of shedding of MICA and/or MICB, an anti-MICA and/or MICB antibody, an MICA and/or MICB down-regulator. For example, the therapeutic agent can be configured comprising IgG1 antibody CLN-619 (Wang, et al., 2023) and/or DM919.

In some embodiments, the therapeutic agent comprises a modulator of tumor necrosis factor (TNF)-related apoptosis-inducing ligand (TRAIL)-mediated apoptosis decoy receptor DcR3 (decoy receptor 3) and/or whereby DcR3 is decreased by an execution of step (1). According to some aspects, DcR3 is decreased dependently of the p53 pathway. In this example, the modulator can comprise a small molecule inhibitor of DcR3 or an antibody inhibitor of DcR3.

In some embodiments, step (1) can induce EIF2-alpha phosphorylation, the central protein involved in activating the integrated stress response (ISR).

In some embodiments, step (1) can increase expression of surface calreticulin, an endoplasmic reticulum stress marker, which activates the ISR.

It is contemplated that the cancer cells, in various embodiments, can include about any known cancer cells, in particular solid tumor cancer cells; examples are glioblastoma cells, mesothelioma cells, thyroid cancer cells, renal cancer cells, ovarian cancer cells, hepatocellular cancer cells, pancreatic cancer cells, lung cancer cells, breast cancer cells, or a combination thereof.

In some embodiments, step (1) includes a downregulation of transcripts coding for one or more enzymes involved in the Krebs cycle, fatty acid synthesis, and/or glycolysis.

According to some aspects, a method for screening a therapeutic agent for synergistic use with TTFields or method for measuring the effect(s) of an alternating electric field upon a cancer cell is disclosed herein, the method comprising the steps of: (1) applying an alternating electric field or tumor treating fields (TTFields) to the cell; (1b) measuring a change in a transcriptome and/or a secretome of one or more cancer cells from the cancer from execution of step (1); and (2) determining a therapeutic agent, a dose and/or a treatment regimen of the agent, to be administered to the subject, using the measurement from step (1b).

In some embodiments, the measuring of transcriptome and/or secretomes of cells includes RNA-seq, multiplexed cytokine profiling, observations of changes in the observable electromagnetic, an analysis by analytical chemistry techniques, PCR, cell-culture, or a combination thereof.

The methods disclosed herein, in some embodiments, can be wherein the cancer cell or cancer is in a solid tumor. The method can be executed wherein an alternating electric field is applied at a frequency in a range of from about 50 kHz to about 1.5 MHz; and the alternating electric field has a field strength of at least about 1 V/cm in at least a portion of the cancer cells or cancer. In some embodiments, the field strength is about a value in the range from about 0.1 V/cm to about 20 V/cm, with all points subsumed. According to some aspects, the alternating electric field or TTFields include an electric field in the range from about 100 kHz to about 500 kHz or in the range from about 100 kHz to about 300 kHz.

In some embodiments, in an Invention brief summary or discussion, the technology disclosed herein can be discussed by reviewing/discussing the following list of features, in which any composition can be described as a method or vice versa, and which can be inter-combined with any other embodiment, example, or aspect disclosed herein:

Feature 1: A composition for use in a method of reducing viability of cancer cells, wherein the method is comprising the steps of: (1) applying an alternating electric field to the cancer cells for a period of time; and (2) administering at least one composition to the cancer cells, wherein the at least one composition comprises at least one inhibitor of at least one immunomodulatory cytokine selected from the group consisting of macrophage migration inhibitory factor (MIF), MHC class I chain-related polypeptide A (MICA), and/or MHC class I chain-related polypeptide B (MICB).

Feature 2: The composition of feature 1, wherein at least one of: the alternating electric field is applied at a frequency in a range of from about 50 kHz to about 1 MHz; the alternating electric field has a field strength of at least about 1 V/cm in at least a portion of the cancer cells; and the period of time that the alternating electric field is applied is at least about 50% of a 24 consecutive hour time period.

Feature 3: The composition of feature 1 or 2, wherein the at least one immunomodulatory cytokine inhibitor comprises an anti-MICA/MICB antibody.

Feature 4: The composition of feature 3, wherein the anti-MICA/MICB antibody comprises one or more of CLN-619 and DM919.

Feature 5: The composition of any one of features 1-4, wherein the at least one immunomodulatory cytokine inhibitor comprises a small molecule inhibitor of MIF.

Feature 6: The composition of feature 5, wherein the small molecule inhibitor of MIF is selected from the group consisting of ISO-1, 4-IPP, Ibudilast, and combinations thereof.

Feature 7: The composition of any one of features 1-6, wherein the at least one immunomodulatory cytokine inhibitor comprises an anti-MIF antibody.

Feature 8: The composition of feature 7, wherein the anti-MIF antibody is selected from the group consisting of RTL100, BAX69, NbE-10, and combinations thereof.

Feature 9: The composition of any one of features 1-8, wherein at least one of steps (1) and (2) is repeated one or more times.

Feature 10: The composition of any one of features 1-9, wherein the cancer cells are selected from the group consisting of hepatocellular carcinoma cells, glioblastoma cells, pleural mesothelioma cells, differentiated thyroid cancer cells, advanced renal cell carcinoma cells, ovarian cancer cells, pancreatic cancer cells, lung cancer cells, breast cancer cells, and combinations thereof.

Feature 11: A composition for use in a method of treating cancer in a subject, wherein the method is comprising the steps of: (1) applying an alternating electric field to a target region of the subject for a period of time; and (2) administering at least one composition to the subject, wherein the at least one composition comprises at least one inhibitor of at least one immunomodulatory cytokine selected from the group consisting of macrophage migration inhibitory factor (MIF), MHC class I chain-related polypeptide A (MICA), and/or MHC class I chain-related polypeptide B (MICB).

Feature 12: The composition of feature 11, wherein at least one of: the alternating electric field is applied at a frequency in a range of from about 50 kHz to about 1 MHz; the alternating electric field has a field strength of at least about 1 V/cm in at least a portion of the target region of the subject; and the period of time that the alternating electric field is applied is at least about 50% of a 24 consecutive hour time period.

Feature 13: The composition of feature 11 or 12, wherein the at least one immunomodulatory cytokine inhibitor comprises an anti-MICA/MICB antibody.

Feature 14: The composition of feature 13, wherein the anti-MICA/MICB antibody comprises one or more of CLN-619 and DM919.

Feature 15: The composition of any one of features 11-14, wherein the at least one immunomodulatory cytokine inhibitor comprises a small molecule inhibitor of MIF.

Feature 16: The composition of feature 15, wherein the small molecule inhibitor of MIF is selected from the group consisting of ISO-1, 4-IPP, Ibudilast, and combinations thereof.

Feature 17: The composition of any one of features 11-16, wherein the at least one immunomodulatory cytokine inhibitor comprises an anti-MIF antibody.

Feature 18: The composition of feature 17, wherein the anti-MIF antibody is selected from the group consisting of RTL100, BAX69, NbE-10, and combinations thereof.

Feature 19: The composition of any one of features 11-18, wherein at least one of steps (1) and (2) is repeated one or more times.

Feature 20: The composition of any one of features 11-19, wherein the cancer is selected from the group consisting of hepatocellular carcinoma, glioblastoma, pleural mesothelioma, differentiated thyroid cancer, advanced renal cell carcinoma, ovarian cancer, pancreatic cancer, lung cancer cell, breast cancer, and combinations thereof.

Feature 21: The composition of any one of features 11-20, further defined as a method of reducing a volume of a tumor and/or preventing an increase of volume of the tumor, wherein the tumor is present in a body of a living subject and includes a plurality of cancer cells.

Feature 22: A composition suitable to be combined with tumor treating fields (TTFields) for use in a method, wherein the method is comprising the steps of: (1) applying an alternating electric field to a target region of the subject for a period of time; and (2) administering at least one above composition from above to the subject, wherein the at least one composition comprises at least one inhibitor of at least one immunomodulatory cytokine selected from the group consisting of macrophage migration inhibitory factor (MIF), MHC class I chain-related polypeptide A (MICA), and/or MHC class I chain-related polypeptide B (MICB); wherein administration of the alternating electric field increases the toxicity of the at least one first composition against cancer cells in the subject when compared to the administration of the at least one composition to the subject in the absence of alternating electric field application.

Feature 23: A method for using TTFields to locate a most effective therapeutic agent and optionally for use in a method for inducing an enhanced cytostatic and/or cytotoxic effect on a cancer cell, wherein the method is comprising the steps of: (1) applying an alternating electric field or tumor treating fields (TTFields) to the cancer cell; (1b) measuring one or more of: an alteration in transcriptional signatures of the cancer cell's cellular metabolism, a modulation in the cancer cell's immune-related cytokines dependent and/or independent of P53, and/or a modulation of the integrated stress response (ISR) either of the cancer cell or a cell in a vicinity of the cancer cell; and (2) selecting a therapeutic agent using the one or more measurements from step (1b) and contacting the cancer cell with said therapeutic agent; whereby an enhanced effect occurs such that the cytostatic and/or cytotoxic effect on the cancer cell is greater than compared to the cytostatic and/or cytotoxic effect, under the same conditions, that occurs from only the execution of step (1).

Feature 24: The method using TTFields of feature 23, wherein step (1b) comprises: (1b) measuring a transcriptome and/or a secretome of the cancer cell after or during execution of step (1), and whereby the measuring is further operative to provide a data indicative for selecting a therapeutic agent to exploit an alteration of the cancer cell by the TTFields or alternating electric field.

Feature 25: The method with TTFields of feature 23 or feature 24, further comprising the step of: (0b) measuring a transcriptome and/or a secretome of the cell before execution of step (1).

Feature 26: The method using TTFields of feature 25, wherein step (1b) is executed after execution of step (1) and one or more changes induced in the transcriptome and/or secretome of the cancer cell after the application of the TTFields are determined via a comparing of a data measured before execution of step (1) (i.e., the data from 0b or a pre-treatment data) and a data measured after execution of step (1) (i.e., the data from 1b or a post-treatment data).

Feature 27: The method using TTFields of feature 23, wherein the method further comprises the steps of using the therapeutic agent for use in a method of treating a subject in need of a cancer treatment thereof, whereby the following method steps are executed: (0) obtaining a subject in need of a cancer treatment, subject suspected as having a need for a cancer treatment, or subject in need of a diagnosis for a cancer treatment; (0b) measuring a transcriptome and/or a secretome of the cancer (or suspected cancer) whereby a pre-treatment data is obtained; (1) applying an alternating electric field or tumor treating fields (TTFields) to the cancer (or to the suspected cancer area); (1b) measuring a transcriptome and/or a secretome of the cancer (or suspected cancer area) after or during execution of step (1), whereby a post-treatment data is obtained; (2) contacting the cancer (or suspected cancer area) with a therapeutic agent, before, after, or during an execution of step (1); wherein the contacting is done by an administration of a therapeutic agent to the subject in need thereof, or an administration of a suitable pharmaceutical formulation, salt, or hydrate thereof; and whereby the subject is treated more effectively for the cancer (i.e., the extent the cancer is stopped or reversed) by a healthcare provider, substantially as described by the method including the steps of (0b), step (1b), and step (2), than compared to the same treatment, of the same subject, by the same method and the same healthcare provider without any execution of any of the steps of step (0b), step (1b), and step (2).

Feature 28: The method with TTFields of feature 26 or feature 27, wherein the pre-treatment data and/or the post-treatment data is/are compared and/or utilized to determine the therapeutic agent utilized in step (2) and/or a dose or a dose regime of the agent.

Feature 29: The method with TTFields of feature 23, wherein the order of the execution of the steps is changed, the method is repeated or wherein the method is executed as a continuous method.

Feature 30: The method with TTFields of feature 27, wherein the pre-treatment and/or post-treatment data is compared to a previously acquired data from a different subject or to a data contained in a database, wherein the comparison includes a machine learning, e-sending to another location, a comparison by a healthcare provider, and/or a storing for future access.

Feature 31: The method with TTFields of feature 23, whereby the cytostatic and/or cytotoxic effects on the cancer cell are greater than when compared to the same effects of applying TTFields (1) without contacting the cancer cell with the therapeutic agent (2) or vice versa; whereby the cytostatic and/or cytotoxic effects on the cancer cell are greater than when compared to contacting the cancer cell with the therapeutic agent (2) without applying the TTFields (1).

Feature 32: The method with TTFields of feature 27, wherein the cancer cell is a cell of a subject in need of a treatment thereof and the method of feature 5 further induces an anti-tumor immunity or an immune response against a cancer cell in the subject.

Feature 33: The method with TTFields of feature 23 or feature 27, whereby execution of step (1) induces a change, upregulation, downregulation, or modulation in a transcriptome and/or a secretome of the cancer (cell) and whereby step (2) either upregulates or downregulates said change.

Feature 34: The method with TTFields of feature 33, wherein the therapeutic agent comprises a modulator of macrophage migration inhibitory factor (MIF) immunomodulatory cytokine.

Feature 35: The method with TTFields of feature 33, whereby MIF is upregulated independently of the p53 pathway by an execution of step (1).

Feature 36: The method with TTFields of feature 34, wherein the modulator comprises a small molecule inhibitor of MIF, an antibody inhibitor of MIF, an anti-MIF antibody, an MIF down-regulator, or an agent that targets MIF receptors.

Feature 37: The method with TTFields of feature 36, wherein the modulator comprises an anti-CD74 monoclonal antibody, Reparixin, ISO-1, 4-IPP, Ibudilast, RTL100, BAX69, NbE-10, a suitable modulator in the field, or a combination thereof.

Feature 38: The method with TTFields of feature 23, wherein the therapeutic agent comprises a modulator of Interleukin 8 (IL-8) and/or whereby IL-8 is downregulated by an execution of step (1).

Feature 39: The method with TTFields of feature 38, wherein the modulator comprises a small molecule inhibitor of IL-8, an antibody inhibitor of IL-8, or an IL-8 down-regulator.

Feature 40: The method with TTFields of feature 38, wherein IL-8 is downregulated independently of the p53 pathway.

Feature 41: The method with TTFields of feature 23, wherein the therapeutic agent comprises a modulator of major histocompatibility complex (MHC) class I chain related-proteins A (MICA) and/or B (MICB) and whereby MICA and/or MICB are each upregulated by an execution of step (1).

Feature 42: The method with TTFields of feature 41, wherein MICA and/or MICB are each or separately upregulated independently of the p53 pathway.

Feature 43: The method with TTFields of feature 41, wherein the modulator comprises a small molecule inhibitor of MICA and/or MICB, an antibody inhibitor of MICA and/or MICB, an anti-MICA and/or MICB antibody, an MICA and/or MICB down-regulator.

Feature 44: The method with TTFields of feature 43, comprising IgG1 antibody CLN-619 and/or DM919.

Feature 45: The method with TTFields of feature 23, wherein the therapeutic agent comprises a modulator of tumor necrosis factor (TNF)-related apoptosis-inducing ligand (TRAIL)-mediated apoptosis decoy receptor DcR3 (decoy receptor 3) and/or whereby DcR3 is decreased by an execution of step (1).

Feature 46: The method with TTFields of feature 44, wherein DcR3 is decreased dependently of the p53 pathway.

Feature 47: The method with TTFields of feature 44, wherein the modulator comprises a small molecule inhibitor of DcR3 or an antibody inhibitor of DcR3.

Feature 48: The method with TTFields of feature 23 wherein step (1) induces EIF2-alpha phosphorylation, the central protein involved in activating the integrated stress response (ISR).

Feature 49: The method with TTFields of feature 23, wherein step (1) increases expression of surface calreticulin, an endoplasmic reticulum stress marker, which activates the ISR.

Feature 50: The method with TTFields of feature 23, wherein the cancer cell (or cancer) includes one or more cells from an acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, Hodgkin's disease, non-Hodgkin's disease, fibrosarcoma, myxosarcoma, liposarcoma, chondrosarcoma, osteogenic sarcoma, thyroid cancer, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioblastoma, glioma, colorectal cancer, astrocytoma, medulloblastoma, craniopharyngioma, ependymoma, pinealoma, hemangioblastoma, acoustic neuroma, oligodenroglioma, schwannoma, meningioma, melanoma, neuroblastoma, retinoblastoma, or a combination thereof.

Feature 51: The method with TTFields of feature 23, wherein step (1) includes a downregulation of transcripts coding for one or more enzymes involved in the Krebs cycle, fatty acid synthesis, and/or glycolysis.

Feature 52: A use of TTFields for use in a method for screening a therapeutic agent for a synergistic use with TTFields or a method for measuring the effect(s) of (or suspected effects of) an alternating electric field upon a cancer cell, wherein the method is comprising the steps of: (1) applying an alternating electric field or TTFields to the cell; (1b) measuring a change in a transcriptome and/or a secretome of one or more cancer cells from the cancer from execution of step (1); and (2) determining a therapeutic agent, a dose and/or a treatment regimen of the agent, to be administered to the subject, using the measurement from step (1b); and/or: optionally repeating the measuring and therapeutic agent so as to provide a determination of effect(s) of or synergistic use(s) of.

Feature 53: The method with TTFields of any one of feature 52, or of any one of features 23-27, wherein the measuring includes RNA-seq, multiplexed cytokine profiling, observations of changes in the observable electromagnetic field(s), an analysis by analytical chemistry/biochemistry techniques, PCR, cell-culture, or a combination thereof, and/or wherein the measuring is substituted, substantially as is written, in the feature by using any technique known in the art to obtain one or more of a measurement on a cancer cell, performed to measure an alteration in transcriptional signatures of cellular metabolism, measure a modulation in immune-related cytokines dependent and/or independent of P53, and/or to measure a modulation of the ISR.

Feature 54: The method with TTFields of feature 53, wherein the cancer cell or cancer is in a solid tumor or in a portion of a solid tumor.

Feature 55: The method with TTFields of feature 53 or of feature 54, wherein: an alternating electric field (or TTFields) is applied at a frequency in a range of from about 50 kHz to about 1 MHz; and the alternating electric field has a field strength in the range from about 0.5 V/cm to about 2 V/cm in at least a portion of the cancer cells or the cancer.

Feature 56: The method with TTFields of feature 52, wherein the alternating electric field or TTFields include an electric field in the range from about 100 kHz to about 500 kHz or in the range from about 100 kHz to about 300 kHz.

In yet other embodiments, a method of making and/or training a system for TTFields concurrent treatment candidates is provided comprising a system or method discussed in any of the embodiments above and optionally adding one or more acceptable training patient datasets. As will be discussed in more detail below, the technology disclosed herein is surprisingly effective and solves many of the larger problems in saving lives discussed herein.

This Brief Summary presents some embodiments as discussed above, while the conceptually large and full inventive concepts are explained and grasped by a study of the entire disclosure (e.g., Detailed Description, FIGS. 1-4, Examples, Brief Summary, Abstract, and the Claims). These and other features and advantages will be apparent from a reading of the following detailed description and a review of the associated figure. It is to be understood that both the foregoing general description and the following detailed description are explanatory only and are not restrictive of aspects as claimed.

Any embodiment, feature, aspect, description, illustration, and/or example herein can be inter-combined with any other in the global spirit/purpose of saving lives. Other implementations are also described and recited herein. These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

Solely for the purpose of illustration, certain embodiments of the present invention are explained using examples in the drawings described below. It should be understood, however, that the invention is not limited to the precise arrangements, dimensions, and configurations shown. In the figures:

FIG. 1 is an example of a method 10 for inducing cytostatic and/or cytotoxic effects on a cancer cell 20 (or for treating a cancer).

FIG. 2 shows a plot of cytokine profiling results comparing principal components (e.g., PCA).

FIG. 3A and FIG. 3B show plots illustrating example cytokines raised by TTFields treatment.

FIG. 3C and FIG. 3D show plots of example cytokines lowered by TTFields treatment.

FIG. 4 shows a plot introducing RNA-seq data with PC 1 on the X-axis and PC 2 on the Y-axis.

Any trademarks, images, likenesses, words, and depictions in the drawings and the disclosure are plainly in fair use and are provided solely for the purposes of illustration of the invention in view of an urgent need to treat subjects as further discussed in detail below. Any additional references can be included in entirety by mentioning herein.

### DETAILED DESCRIPTION OF THE INVENTION

In some discussions, the subject innovation is now described with increasing details. In the following description, for purposes of explanation, specific details are set forth in order to provide an understanding of the present invention. It may be evident, however, that the present invention may be practiced without these specific details. In particular, it may become so evident after the conceptually large inventive concepts are grasped in a mind of a skilled operator/artisan. In other instances, the inventive concepts are described with Examples to provide insights into the hope inspiring and life-saving benefits of the invention.

It is to be appreciated that certain aspects, modes, embodiments, variations and features of the invention are described below in various levels of detail in order to provide a substantial understanding of the present invention.

The following description of particular aspect(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the compositions or processes are described as using specific materials or an order of individual steps, it is appreciated that materials or steps may be interchangeable such that the description of the invention may include multiple parts or steps arranged in many ways as is readily appreciated by one of skill in the art.

Before explaining at least one detailed embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (e.g., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

The term “therapeutic composition” or “pharmaceutical composition” as used herein refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (e.g., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one condition, disease, and/or infection. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “concomitant therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered at the same time, within one minute, 2 minutes, 4 minutes, 6 minutes, 10 minutes, 30 minutes, or an hour or 90 minutes of one another. In some embodiments, the different therapeutic compounds can be administered within 1 year of one another. Thus, an individual who receives such treatment can benefit from a combined effect of different therapeutic compounds.

As used herein, the terms “treat,” “treatment,” “treating,” or “amelioration” when used in reference to a disease, disorder, or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), sign(s), diminishment of extent of the deficit, stabilized (i.e., not worsening) state of a symptom or condition, delay or slowing of onset of symptoms or indications, and an increased lifespan as compared to that expected in the absence of treatment.

As used herein, the term “long-term” administration means that the therapeutic agent or drug is administered for a period of at least 12 weeks. This includes that the therapeutic agent, combination, or drug is administered such that it is effective over, or for, a period of at least 12 weeks and does not necessarily imply that the administration itself takes place for 12 weeks, e.g., if sustained release compositions or long-acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by aerosol inhalation, injection, ingestion, transfusion, implantation, application (e.g., topical, otic, or ocular), or transplantation. Administration can be accomplished by an implant. In some embodiments, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into an artery, vein, lymph node, or organ (e.g., heart, muscle, organ).

The terms: “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The term “prodrug” is intended to encompass compounds which, under physiologic conditions, are converted into the therapeutically active agents of the present invention (e.g., any compound selected from this disclosure). A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds selected from this disclosure in a formulation can be replaced with the corresponding suitable prodrug, e.g., wherein a hydroxyl in the parent compound is presented as an ester or a carbonate or carboxylic acid present in the parent compound is presented as an ester. In some embodiments, a “prodrug” is made by using an absorbing particle that subsequently releases an active form after administration.

In some embodiments, the decrease in the one or more signs or symptoms is evaluated according to the DSM-5. In some embodiments, signs are observed or measured by a health care provider. Symptoms can be reported by the subject. In some embodiments, the decrease of signs or symptoms occurs in less than about 120 minutes, 90 minutes, less than about 60 minutes, less than about 30 minutes, less than about 15 minutes, less than about 10 minutes, or less than about 5 minutes, or less than about 3 minutes, or less than about 1 minute. In some embodiments, the decrease of signs or symptoms occurs in less than 1 day, less than 1 week, less than 1 month, or in less than 1 year.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, an agent or a therapeutic agent provided to a subject and suspected to be or involved in a treatment can be a small molecule less than 1000 MW or a large molecule not less than 1000 MW including, for example, biologics, oligonucleotides, peptides, systems of large molecules, oligosaccharides, and larger molecules. Any of the therapeutic agents disclosed herein can be used as or in combination with small molecules and/or large molecules as discussed herein.

As used herein, a subject may or may not be aware of suffering from a cancer or a disease condition. A health care provider may suspect a disease or cancer or may have confirmed cancer or disease.

A subject can be one who has been previously diagnosed with or identified as suffering from or having a condition in need of treatment (e.g., a cancer or related disorder) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. For example, a subject can be one who exhibits one or more risk factors for a condition, or one or more complications related to a condition or a subject who does not exhibit risk factors. A “subject in need” of treatment for a particular condition can be a subject having that condition, diagnosed as having that condition, suspected as having, or at risk of developing that condition. In another example, the subject has been brought into a treatment situation entirely without the subject's knowledge and/or intent. For example, a subject can obviously be in need of treatment but not be responsive to a treatment, and as described herein the present methods and formulations may be used to help save the subject's life.

The compositions and methods of the present invention may be utilized to treat an individual (or subject) in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973; 5,763,493; 5,731,000; 5,541,231; 5,427,798; 5,358,970; and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragées, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragées, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropyl methyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, micro-emulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chloro-butanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher, et al., 1996).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In other embodiments, the active compound will be administered once daily.

The patient receiving this treatment is any animal in need, including primates, in particular humans; and other mammals such as equines bovine, porcine, sheep, feline, and canine; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, I-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

The amounts effective can be determined with no more than routine experimentation. For example, amounts effective may range from about 1 ng/kg to about 200 mg/kg, about 1 ag/kg to about 100 mg/kg, or about 1 mg/kg to about 50 mg/kg. The dosage of a composition can be at any dosage including, but not limited to, about 1 ag/kg. The dosage of a composition may be at any dosage including, but not limited to, about 1 ag/kg, about 10 ag/kg, about 25 ag/kg, about 50 ag/kg, about 75 ag/kg, about 100 ag/kg, about 125 ag/kg, about 150 ag/kg, about 175 ag/kg, about 200 ag/kg, about 225 ag/kg, about 250 ag/kg, about 275 ag/kg, about 300 ag/kg, about 325 ag/kg, about 350 ag/kg, about 375 ag/kg, about 400 ag/kg, about 425 ag/kg, about 450 ag/kg, about 475 ag/kg, about 500 ag/kg, about 525 ag/kg, about 550 ag/kg, about 575 ag/kg, about 600 ag/kg, about 625 ag/kg, about 650 ag/kg, about 675 ag/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. In other embodiments, the dosage is 1 mg-500 mg. In some embodiments, the dosage is 10, 20, 30, 40, 50, 60, 70, 80, 90, 100, 110, 120, 130, 140, or 150 mg. These doses may be unitary or divided and may be administered one or more times per day. The above dosages are exemplary of the average case, but there can be individual instances in which higher or lower dosages are merited, and such are within the scope of this disclosure. In practice, the physician determines therapeutically effective amounts and the actual dosing regimen that is most suitable for an individual subject, which can vary with the age, weight, and response of the particular subject.

The therapeutic agents may be administered once, twice or three times per day for 1 day to the end of life, or for 1 day to 1, 2, 3, 4, 5, 6, 7, 8, 9, or 10 or more years, or until the agents cause unacceptable side effects or are no longer useful.

The patient is generally monitored for changes in the symptoms. In one embodiment, there is a reduction in the symptoms. In another embodiment, the symptoms remain about the same and there is no evidence of progression. Methods for monitoring and quantifying any change of these symptoms can be carried out by routine methods or by routine experimentation.

Any change in symptoms may be monitored for 1-36 months or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months.

In another embodiment, the patient is monitored for a change in the underlying pathology. In one embodiment, there is a reduction in the underlying pathology. In another embodiment, the underlying pathology remains about the same and there is no evidence of progression.

In some embodiments, any change in the underlying pathology is identified by detection of a biomarker before and after the administration of the TTFields. In one embodiment, the biomarker is determined by measuring a transcriptome and/or secretome. In another embodiment, the biomarker is detected by PET imaging. In another embodiment, the underlying pathology is identified by measurement of a tumor volume before and after the TTFields administration.

In some embodiments, the decrease of the underlying pathology is reversed or delayed for at 1-36 months, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the first administration of TTFields.

In some embodiments, the patient is also administered at least one second therapeutic agent useful for the treatment. The at least one second therapeutic agent may be administered separately or together as part of a unitary pharmaceutical composition.

The patient may be monitored for improvement of symptoms. Such symptoms can include one or more of the following: difficulty walking or doing normal daily activities, tripping and falling, weakness of the legs, feet or ankles, hand weakness or clumsiness, slurred speech or trouble swallowing, muscle cramps, twitching in the arms, shoulders or tongue, inappropriate crying, cognitive changes, and behavior changes.

Any change in symptoms may be monitored for 1-36 months or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In some embodiments, the decrease of the underlying pathology is reversed or delayed for at 1-36 months or longer, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the first administration of treatments herein.

Antibody drug conjugates (ADCs) can be utilized to delivery one or more therapeutic agents disclosed herein. For example, an antibody that has affinity for a tumor can be attached to a linker, which is attached to a therapeutic agent. The linker can be, for example, a self-immolating linker or a linker designed to provide a release via an enzymatic or hydrolytic mechanism.

### Further Example Salts, Pharmaceutical Compositions, and Kits

The methods of the present disclosure can be accomplished by administering a therapeutic agent as the neat compound or as a pharmaceutical composition. Administration of a pharmaceutical composition or neat compound can be performed before or after the clinical diagnosis of a disorder associated with cancer. Typically, the pharmaceutical compositions are sterile and contain nothing that would cause an adverse reaction when administered.

Further provided are kits comprising at least one therapeutic agent, and, optionally, at least one second therapeutic agent useful for the treatment or prevention of a disorder associated with cancer, packaged separately or together, and an insert having instructions for using these active agents. In one embodiment, the at least one therapeutic agent is packaged alone together with instructions to administered together with the at least one second therapeutic agent. The at least one first agent and the at least one second therapeutic agent can be administered simultaneously or sequentially to achieve the desired effect. In addition, first therapeutic agent and the at least one second therapeutic agent can be administered from a single composition or two separate compositions.

The second therapeutic agent is administered in an amount to provide its desired therapeutic effect. The effective dosage range for each optional therapeutic agent is known in the art, and the optional therapeutic agent is administered to an individual in need thereof within such established ranges.

The present disclosure encompasses the preparation and use of salts of therapeutic agents. As used herein, a “pharmaceutically acceptable salt” refers to salts or zwitterionic forms of the therapeutic agents. Salts of therapeutic agents can be prepared during the final isolation and purification of the compounds or separately by reacting the compound with a suitable acid. The pharmaceutically acceptable salts of therapeutic agents can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Non-limiting examples of salts of therapeutic agents include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2 naphthalenesulfonate, oxalate, pamoate, pectinate, persulfate, 3 phenylproprionate, picrate, pivalate, propionate, trichloroacetate, trifluoroacetate, phosphate, glutamate, bicarbonate, para-toluenesulfonate, undecanoate, lactate, citrate, tartrate, gluconate, methanesulfonate, ethanedisulfonate, benzene sulfonate, and p toluenesulfonate salts. In addition, available amino groups present in the therapeutic agents can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to “therapeutic agents” appearing herein is intended to include the “therapeutic agents” as well as pharmaceutically acceptable salts, hydrates, or solvates thereof.

The present disclosure encompasses the preparation and use of solvates of therapeutic agents. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g., a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. therapeutic agents can be present as solvated forms with a pharmaceutically acceptable solvent, such as water, methanol, and ethanol, and it is intended that the disclosure includes both solvated and unsolvated forms of therapeutic agents. One type of solvate is a hydrate. A “hydrate” relates to a particular subgroup of solvates where the solvent molecule is water. Solvates typically can function as pharmacological equivalents. Preparation of solvates is known in the art. See, e.g., Caira et al. (2004), which describes the preparation of solvates of fluconazole with ethyl acetate and with water. Similar preparation of solvates, hemisolvates, hydrates, and the like are described by Van Tonder et al. (2004) and Bingham et al. (2001). A typical, non-limiting, process of preparing a solvate would involve dissolving the at least one therapeutic agent or at least one second therapeutic agent in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvate in a crystal of the solvate. For example, a therapeutic agent is dissolved in water or methanol, dried, and the resulting O—H stretch (from the formation of the solvate/hydrate) is confirmed by an ATR-IR measurement. The O—H stretch is not found in the non-solvate/hydrate form, quickly confirming the formation of the solvate/hydrate in the solid form by the ATR-IR measurement.

The at least one therapeutic agent and at least one second therapeutic agent typically are administered in admixture with a pharmaceutical carrier to give a pharmaceutical composition selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of the at least one therapeutic agent and at least one second therapeutic agent.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the at least one therapeutic agent and/or at least one second therapeutic agent is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50% of the at least one therapeutic agent and at least one second therapeutic agent. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of the at least one therapeutic agent and at least one second therapeutic agent.

When a therapeutically effective amount of the at least one therapeutic agent and at least one second therapeutic agent is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

The at least one therapeutic agent and at least one second therapeutic agent can be readily combined with pharmaceutically acceptable carriers well-known in the art. Standard pharmaceutical carriers are described in Remington's Pharmaceutical Sciences, 1995. Such carriers enable the active agents to be formulated as tablets, pills, dragees, capsules, liquids, gels, syrups, slurries, suspensions and the like, for oral ingestion by a subject to be treated. Pharmaceutical preparations for oral use can be obtained by adding the at least one therapeutic agent and/or the at least one second therapeutic agent to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

The at least one therapeutic agent and at least one second therapeutic agent can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of the at least one therapeutic agent and at least one second therapeutic agent can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

The at least one therapeutic agent and at least one second therapeutic agent also can be formulated in rectal compositions, such as suppositories or retention enemas, e.g., containing conventional suppository bases. In addition to the formulations described previously, the at least one therapeutic agent and at least one second therapeutic agent also can be formulated as a depot preparation. Such long-acting formulations can be administered by implantation (for example, subcutaneously or intramuscularly) or by intramuscular injection. Thus, for example, the at least one therapeutic agent and at least one second therapeutic agent can be formulated with suitable polymeric or hydrophobic materials (for example, as an emulsion in an acceptable oil) or ion exchange resins.

In particular, the at least one therapeutic agent and at least one second therapeutic agent can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. The at least one therapeutic agent and at least one second therapeutic agent also can be injected parenterally, for example, intravenously, intramuscularly, subcutaneously, or intra-coro-narily. For parenteral administration, the at least one therapeutic agent and at least one second therapeutic agent are typically used in the form of a sterile aqueous solution which can contain other substances, for example, salts or monosaccharides, such as mannitol or glucose, to make the solution isotonic with blood.

In particular referral to the Definitions used herein, a “small molecule” refers to a chemical that has a molecular weight of 1000 or less in a free form (i.e., as measured in a non-salt form). A reference to a “large molecule” refers to a molecular weight of greater than 1000.

It should be understood that any of the definitions in the examples above do not limit the spirit of the Invention, namely, to save lives.

Example Compositions, Systems, and Methods for Treating Cancer Using Tumor Treating Fields with Inhibitors of MIF, Mica, and/or MICB

Turning now to examples of novel and inventive concept(s), a concurrent therapy for cancer is disclosed herein. The concurrent therapy includes the use of alternating electric fields (e.g., TTFields) in addition with at least one inhibitor of an immunomodulatory cytokine, such as (but not limited to) macrophage migration inhibitory factor (MIF), MHC class I chain-related polypeptide A (MICA), and/or MHC class I chain-related polypeptide B (MICB).

Certain non-limiting embodiments of the present disclosure are directed to a method of reducing viability of cancer cells. The method includes the steps of: (1) applying an alternating electric field to the cancer cells for a period of time; and (2) administering at least one composition to the cancer cells, wherein the at least one composition comprises at least one inhibitor of at least one immunomodulatory cytokine, such as (but not limited to) MIF, MICA, and/or MICB. According to some aspects, this is correctly phrased as inhibition of MICA/B shedding

Certain additional non-limiting embodiments of the present disclosure are directed to a method of treating cancer in a subject. The method includes the steps of: (1) applying an alternating electric field to a target region of the subject for a period of time; and (2) administering at least one composition to the subject, wherein the at least one composition comprises at least one inhibitor of at least one immunomodulatory cytokine, such as (but not limited to) MIF, MICA, and/or MICB.

Certain additional non-limiting embodiments of the present disclosure are directed to a method of reducing a volume of a tumor present in a body of a living subject, wherein the tumor includes a plurality of cancer cells. The method includes the steps of: (1) applying an alternating electric field to a target region of the subject for a period of time; and (2) administering at least one composition to the subject, wherein the at least one composition comprises at least one inhibitor of at least one immunomodulatory cytokine, such as (but not limited to) MIF, MICA, and/or MICB.

Certain additional non-limiting embodiments of the present disclosure are directed to a method of preventing an increase of volume of a tumor, wherein the tumor is present in a body of a living subject and includes a plurality of cancer cells. The method includes the steps of: (1) applying an alternating electric field to a target region of the subject for a period of time; and (2) administering at least one composition to the subject, wherein the at least one composition comprises at least one inhibitor of at least one immunomodulatory cytokine, such as (but not limited to) MIF, MICA, and/or MICB.

Certain additional non-limiting embodiments of the present disclosure are directed to a method, comprising the steps of: (1) applying an alternating electric field to a target region of the subject for a period of time; and (2) administering at least one composition to the subject, wherein the at least one composition comprises at least one inhibitor of at least one immunomodulatory cytokine, such as (but not limited to) MIF, MICA, and/or MICB. In a particular (but non-limiting) embodiment, administration of the alternating electric field increases the toxicity of the at least one composition against cancer cells in the subject when compared to the administration of the at least one composition to the subject in the absence of alternating electric field application.

The steps of any of the methods of the present disclosure may be performed concomitantly or serially, and in particular, substantially simultaneously or wholly or partially sequentially.

The methods of the present disclosure may be utilized to treat any types of cancer cells/cancers/tumors that respond to treatment with alternating electric fields (e.g., TTFields) and/or immunomodulatory cytokine inhibitors. Non-limiting examples of cancer cells/cancers/tumors that can be treated in accordance with the present disclosure include hepatocellular carcinoma/carcinoma cells, glioblastoma/glioblastoma cells, pleural mesothelioma/mesothelioma cells, differentiated thyroid cancer/cancer cells, advanced renal cell carcinoma/carcinoma cells, ovarian cancer/cancer cells, pancreatic cancer/cancer cells, lung cancer/cancer cells, breast cancer/cancer cells, and the like, as well as any combination thereof.

Any type of conductive or non-conductive electrode(s) and/or transducer array(s) that can be utilized for generating an alternating electric field that are known in the art or otherwise contemplated herein may be utilized for generation of the alternating electric field in accordance with the methods of the present disclosure. Non-limiting examples of electrodes and transducer arrays that can be utilized for generating an alternating electric field in accordance with the present disclosure include those that function as part of a TTFields system as described, for example but not by way of limitation, in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016.

The alternating electric field may be generated at any frequency in accordance with the present disclosure. For example (but not by way of limitation), the alternating electric field may have a frequency of about 50 kHz, about 75 kHz, about 100 kHz, about 125 kHz, about 150 kHz, about 175 kHz, about 200 kHz, about 225 kHz, about 250 kHz, about 275 kHz, about 300 kHz, about 325 kHz, about 350 kHz, about 375 kHz, about 400 kHz, about 425 kHz, about 450 kHz, about 475 kHz, about 500 kHz, about 550 kHz, about 600 kHz, about 650 kHz, about 700 kHz, about 750 kHz, about 800 kHz, about 850 kHz, about 900 kHz, about 950 kHz, about 1 MHz, about 2 MHz, about 3 MHz, about 4 MHz, about 5 MHz, about 6 MHz, about 7 MHz, about 8 MHz, about 9 MHz, about 10 MHz, and the like, as well as a range formed from any of the above values (e.g., a range of from about 50 kHz to about 10 MHz, a range of from about 50 kHz to about 1 MHz, a range of from about 50 kHz to about 500 kHz, a range of from about 100 kHz to about 500 kHz, a range of from about 150 kHz to about 300 kHz, etc.), and a range that combines two integers that fall between two of the above-referenced values (e.g., a range of from about 122 kHz to about 313 kHz, a range of from about 78 kHz to about 298 kHz, etc.).

In certain particular (but non-limiting) embodiments, the alternating electric field may be imposed at two or more different frequencies. When two or more frequencies are present, each frequency is selected from any of the above-referenced values, or a range formed from any of the above-referenced values, or a range that combines two integers that fall between two of the above-referenced values.

The alternating electric field may have any field strength in the subject/cancer cells, so long as the alternating electric field is capable of functioning in accordance with the present disclosure. For example (but not by way of limitation), the alternating electric field may have a field strength of at least about 1 V/cm, about 1.5 V/cm, about 2 V/cm, about 2.5 V/cm, about 3 V/cm, about 3.5 V/cm, about 4 V/cm, about 4.5 V/cm, about 5 V/cm, about 5.5 V/cm, about 6 V/cm, about 6.5 V/cm, about 7 V/cm, about 7.5 V/cm, about 8 V/cm, about 9 V/cm, about 9.5 V/cm, about 10 V/cm, about 10.5 V/cm, about 11 V/cm, about 11.5 V/cm, about 12 V/cm, about 12.5 V/cm, about 13 V/cm, about 13.5 V/cm, about 14 V/cm, about 14.5 V/cm, about 15 V/cm, about 15.5 V/cm, about 16 V/cm, about 16.5 V/cm, about 17 V/cm, about 17.5 V/cm, about 18 V/cm, about 18.5 V/cm, about 19 V/cm, about 19.5 V/cm, about 20 V/cm, and the like, as well as a range formed from any of the above values (e.g., a range of from about 1 V/cm to about 20 V/cm, a range of from about 1 V/cm to about 10 V/cm, a range of from about 1 V/cm to about 4 V/cm, etc.), and a range that combines two integers that fall between two of the above-referenced values (e.g., a range of from about 1.1 V/cm to about 18.6 V/cm, a range of from about 1.2 V/cm to about 9.8 V/cm, a range of from about 1.3 V/cm to about 4.7 V/cm, etc.).

In some instances, the electric field in at least a portion of the target region/subject/cancer cells is induced by an applied voltage that is determined by computer simulation of the target region/subject/cancer cells. In some instances, the electric field in at least a portion of the target region/subject/cancer cells is induced by an applied current of at least 0.1 Amp, 0.5 Amp, or 1 Amp. In some embodiments, In some instances, an alternating electric field-generating device provides a current of between 0.1 Amp and 10 Amp, 0.5 Amp and 7 Amp, 0.5 Amp and 5 Amp, or 0.9 Amp and 4 Amp.

The alternating electric field may be applied in a single direction between a pair of arrays or may be alternating in two or more directions/channels between two or more pairs of arrays (e.g., front-back and left-right). For example, certain TTFields devices (such as, but not limited to, the OPTUNE® system (Novocure Limited, St. Helier, Jersey)) operate in two directions in order to increase the chances that a dividing cell will be aligned with the electric field such that the electric field can have the desired anti-mitotic effect. However, it will be understood that the scope of the present disclosure also includes the application of the alternating electric field in a single direction. The term “alternating electric field” as used herein will be understood to include application in a single direction/channel as well as in two or more directions/channels; in addition, the term “alternating electric field” as used herein will be understood to include both application of a single alternating electric field as well as application of a plurality of alternating electric fields in succession for a duration of time.

The alternating electric field may be applied for any continuous or cumulative period of time sufficient to achieve a reduction in viability of cancer cells and/or a reduction in tumor volume (and/or a prevention of increase in tumor volume). The period of time that the alternating electric field is applied includes both a continuous period of time as well as a cumulative period of time. That is, the period of time that the alternating electric field is applied includes a single session, which may include minor breaks for changing arrays or charging the signal generator (i.e., continuous application). For example, a subject is allowed to take breaks during treatment with an alternating electric field device and is only expected to have the device positioned on the body and operational (i.e., generating an alternating electric field) for at least about 50%, at least about 60%, at least about 70%, or at least about 80% of a day. In some embodiments, a subject is expected to have the device generating an alternating electric field for at least about 50%, at least about 60%, at least about 70%, or at least about 80% of the total treatment period (e.g., over a course of one day, one week, two weeks, one month, two months, three months, four months, five months, etc.). The period of time that the alternating electric field is applied can also refer to the total number of hours/days/weeks/months etc. on therapy, not accounting for breaks (i.e., cumulative period).

For example, but not by way of limitation, the alternating electric field may be applied for a continuous or cumulative period of time of at least about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, about 8 days, about 9 days, about 10 days, about 11 days, about 12 days, about 13 days, about 14 days, about 21 days, about 1 month, about 2 months, about 3 months, about 4 months, about 5 months, about 6 months, and the like, as well as a range formed from any of the above values (e.g., a range of from about 1 hour to about 6 months, a range of from about 24 hours to about 72 hours, etc.), and a range that combines two integers that fall between two of the above-referenced values (e.g., a range of from about 14 hours to about 68 hours, etc.).

In a particular (but non-limiting) embodiment, the period of time that the alternating electric field is applied is at least about 24 cumulative hours within 48 consecutive hours.

Any compositions that function as an inhibitor of at least one immunomodulatory cytokine that are known in the art or are otherwise contemplated herein may be utilized in accordance with the present disclosure, so long as the compositions are capable of functioning as described herein. Suitable types of immunomodulatory cytokine inhibitors that may be utilized in accordance with the present disclosure include, but are not limited to, MIF inhibitors, MICA inhibitors, and/or MICB inhibitors. The inhibitors may be small molecule inhibitors and/or antibodies.

Particular (but non-limiting) examples of anti-MICA/MICB antibodies that may be utilized in accordance with the present disclosure are the clinically tested MICA/B specific IgG1 antibodies CLN-619 and DM919 (Lucas Ferrari de Andrade, et al. (2020) Cancer Immunol Res, 8(6):769-780; Whalen et al. (2022) Cancer Res, 82(12_Supplement):3506; Guangan Hu et al, J Immunother Cancer 2022; 10(Suppl 2):A1-A1603). In some embodiments, CLN-619 functions by restoring MICA/B expression on the surface of tumor cells, enhancing the interaction between MICA and NKG2D, and inducing antibody-dependent cellular toxicity (ADCC), together promoting anti-tumor activity via NKG2D-expressing NK and T cells. According to this aspect, the technology correctly would be inhibition of MICA/B shedding.

Particular (but non-limiting) examples of small molecule inhibitors of MIF that may be utilized in accordance with the present disclosure include ISO-1, 4-IPP, Ibudilast, CPSI 1360, MIF098, MIF-IN-1, MIF-IN-4, MIF-IN-5, MIF-IN-6, R110, and the like (Wen et al. (2021) Front Pharmacol, 12:638950).

Particular (but non-limiting) examples of anti-MIF antibodies that may be utilized in accordance with the present disclosure include RTL100, BAX69, NbE-10, and the like (Wen et al. (2021) Front Pharmacol, 12:638950).

The composition(s) of the present disclosure may be provided with any formulation known in the art or otherwise contemplated herein. In certain particular (but non-limiting) embodiments, the compositions contain one or more pharmaceutically acceptable carriers (and as such, the composition may also be referred to as a “pharmaceutical composition”). Non-limiting examples of suitable pharmaceutically acceptable carriers include water; saline; dextrose solutions; fructose or mannitol; calcium carbonate; cellulose; ethanol; oils of animal, vegetative, or synthetic origin; carbohydrates, such as glucose, sucrose, or dextrans; antioxidants, such as ascorbic acid or glutathione; chelating agents; low molecular weight proteins; detergents; liposomal carriers; nanocarriers; scaffolds that allowed delayed drug release (such as, but not limited to, hydrogels); buffered solutions, such as sodium chloride, saline, phosphate-buffered saline, and/or other substances which are physiologically acceptable and/or safe for use; diluents; excipients such as polyethylene glycol (PEG); or any combination thereof. Suitable pharmaceutically acceptable carriers for pharmaceutical formulations are described, for example, in Remington: The Science and Practice of Pharmacy, 23rd ed. (2020).

In certain particular (but non-limiting) embodiments, the composition(s) of the present disclosure may further contain one or more additional active agents. Various active agents that can be utilized concurrently with alternating electric fields and/or immunomodulatory cytokine inhibitors are known in the art, and certain combination therapies are approved by the FDA or currently in clinical trials testing.

In addition, any of the compositions of the present disclosure may contain other agents that allow for administration of the compositions via a particular administration route. For example, but not by way of limitation, the compositions may be formulated for administration by oral, topical, transdermal, parenteral, subcutaneous, intranasal, mucosal, intramuscular, intraperitoneal, intravitreal, and/or intravenous routes. Based on the route of administration, the compositions may also contain one or more additional components in addition to the active agent(s) (e.g., immunomodulatory cytokine inhibitor(s) and/or additional therapeutic agent(s)). Examples of additional secondary compounds that may be present include, but are not limited to, fillers, salts, buffers, preservatives, stabilizers, solubilizers, wetting agents, emulsifying agents, dispersing agents, gels, adhesives, and other materials well known in the art.

In a particular (but non-limiting) embodiment, any of the compositions of the present disclosure is administered via injection or implantation into the subject. For example (but not by way of limitation), in some instances, it may be desired that the composition(s) be administered on a local/regional level to ensure targeting of the composition(s) to a specific location in the body of the subject and inhibit non-specific interactions in other parts of the body; in other instances, a more systemic administration may be desired.

Any of the compositions of the present disclosure may be administered before or after application of the alternating electric field has begun. In certain particular (but non-limiting) embodiments, at least one composition may be administered before the application of the alternating electric field has begun. In other particular (but non-limiting) embodiments, the at least one composition may be administered after the application of the alternating electric field has begun. In particular (but not by way of limitation), the composition(s) may be administered during application of the alternating electric field (e.g., before the period of time that the alternating electric field is applied has elapsed) and/or after application of the alternating electric field has elapsed.

For example (but not by way of limitation), any of the compositions of the present disclosure may be administered after application of the alternating electric field has commenced by a period of at least about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the at least one composition is administered at least about 24 hours after application of the alternating electric field has begun.

In a particular (but non-limiting) embodiment, one or more of the compositions of the present disclosure is administered within about 96 hours of when the period of time elapsed.

The compositions of the present disclosure may be administered to the cancer cells/subject at any concentration that provides a therapeutically effective concentration of the active agent(s) (i.e., immunomodulatory cytokine inhibitor(s)). In certain non-limiting embodiments, the application of the alternating electric field reduces the amount of the active agent required to be therapeutically effective when compared to a normal therapeutically effective amount of active agent administered in the absence of an alternating electric field. For example, but not by way of limitation, the therapeutically effective concentration of the composition may be reduced by at least about 25%, about 30%, about 35%, about 40%, about 45%, about 50%, about 55%, about 60%, about 65%, about 70%, about 75% or more with respect to a dosage of the composition known to be therapeutically effective in the absence of application of an alternating electric field. In a particular (but non-limiting) embodiment, the therapeutically effective concentration of the composition is reduced by at least about 50% when compared to a dosage of the composition known to be therapeutically effective in the absence of an alternating electric field.

The therapeutically effective concentration of each active agent utilized in accordance with the present disclosure may be, for example (but not by way of limitation), about 1 nM, about 10 nM, about 20 nM, about 30 nM, about 40 nM, about 50 nM, about 60 nM, about 70 nM, about 80 nM, about 90 nM, about 100 nM, about 125 nM, about 150 nM, about 175 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, and the like, as well as a range formed from any of the above values (e.g., a range of from about 12.5 nM to about 100 nM, a range of from about 1 mM to about 20 mM, etc.), and a range that combines two integers that fall between two of the above-referenced values (e.g., a range of from about 17 nM to about 83 nM, etc.).

In a particular (but non-limiting) embodiment, the therapeutically effective concentration of each active agent is from about 10 nM to about 100 nM.

In particular (but non-limiting) embodiments, the therapeutically effective concentration of each active agent utilized in accordance with the present disclosure may be, for example (but not by way of limitation), about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, and the like, as well as a range formed from any of the above values (e.g., a range of from about 10 mg/kg to about 50 mg/kg, a range of from about 1 mg/kg to about 40 mg/kg, a range of from about 1 mg/kg to about 30 mg/kg, a range of from about 1 mg/kg to about 25 mg/kg, a range of from about 1 mg/kg to about 20 mg/kg, a range of from about 1 mg/kg to about 10 mg/kg, etc.).

In certain particular (but non-limiting) embodiments, the method includes one or more additional steps. For example (but not by way of limitation), the method may further include the step of: discontinuing the application of the alternating electric field (such as, but not limited to, to allow the cells/tissue to recover). In addition, any of the steps may be repeated one or more times.

In certain particular (but non-limiting) embodiments, any of the compositions of the present disclosure may be administered by any dosage regimen known in the art. For example, but not by way of limitation, each composition may be administered in a single dosage or multiple dosages over a defined treatment period. For example (but not by way of limitation), a therapeutically effective concentration of one or more compositions may be administered about once every 4 hours, about once every 8 hours, about once every 12 hours, about once every day, about once every other day, about once every three days, about once a week, about twice a week, about three times a week, about once every two weeks, about once every three weeks, about once a month, and the like, as well as a range formed from any of the above values (a range of about once every 4 to 8 hours, a range of from about once a week to about once a month, etc.).

In addition, when multiple compositions are administered (i.e., two immunomodulatory cytokine inhibitors present in different compositions), the two or more compositions may be administered via the same route (e.g., both administered intravenously), or the two or more compositions may be administered by different routes (e.g., one composition orally administered, and another composition intravenously administered).

In certain particular (but non-limiting) embodiments, the method involves concurrent therapy with yet additional compositions. As such, the method may include an additional step of administering at least one additional therapeutic composition to the cancer cells/subject.

When present, administration of the at least one additional therapeutic composition may be performed substantially simultaneously or wholly or partially sequentially with the administration of any of the composition(s) containing the immunomodulatory cytokine inhibitor(s), whereby the separate compositions are administered simultaneously or wholly or partially sequentially. Also, when the method includes administration of the additional composition, the optional administration step may be performed before or after the application of the alternating electric field has begun, during application of the alternating electric field, and/or after application of the alternating electric field has elapsed, in the same manner(s) and time frame(s) as described above for the other composition(s).

In certain particular (but non-limiting) embodiments, the method may further comprise the step of administering at least one additional therapy to the cells/subject. Any therapies known in the art or otherwise contemplated herein for use with alternating electric fields (e.g., TTFields) and/or immunomodulatory cytokine inhibitors may be utilized in accordance with the methods of the present disclosure. Non-limiting examples of additional therapies that may be utilized include radiation therapy, photodynamic therapy, transarterial chemoembolization (TACE), or combinations thereof.

Any of the method steps may be repeated one or more times. Each of the steps can be repeated as many times as necessary. When the step of applying the alternating electric field is repeated, the transducer arrays may be placed in slightly different positions on the subject than their original placement; relocation of the arrays in this manner may further aid in treatment of the tumor/cancer. In addition, any of the steps of administering any of the compositions/additional therapies may be repeated various times and at various intervals to follow any known and/or generally accepted dosage/treatment regimen for the composition(s)/therapy(ies).

Certain non-limiting embodiments of the present disclosure are related to kits that include any of the components of the alternating electric field (e.g., TTFields) generating systems disclosed or otherwise contemplated herein (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with at least one of any of the compositions disclosed or otherwise contemplated herein. The kits may optionally further include one or more of any of the optional compositions disclosed or otherwise contemplated herein (such as, but not limited to, one or more optional compositions containing at least one additional active agent). The kits may optionally further include one or more devices (or one or more components of devices) utilized in one or more additional therapy steps.

In a particular (but non-limiting) embodiment, the kit may further include instructions for performing any of the methods disclosed or otherwise contemplated herein. For example (but not by way of limitation), the kit may include instructions for applying one or more components of the alternating electric field (e.g., TTFields) generating device to the skin of the patient, instructions for applying the alternating electric field to the patient, instructions for formulating one or more of the compositions, instructions for when and how to administer the one or more compositions, and/or instructions for when to activate and turn off the alternating electric field in relation to the administration of the composition(s) and/or optional therapy steps.

In addition to the components described in detail herein above, the kits may further contain other component(s)/reagent(s) for performing any of the particular methods described or otherwise contemplated herein. For example (but not by way of limitation), the kits may additionally include: (i) components for preparing the skin prior to disposal of the hydrogel compositions and/or transducer arrays thereon (e.g., a razor, a cleansing composition or wipe/towel, etc.); (ii) components for removal of the gel/transducer array(s); (iii) components for cleansing of the skin after removal of the gel/transducer array(s); and/or (iv) other components utilized with the system (e.g., conductive material, nonconductive material, a soothing gel or cream, a bandage, etc.). The nature of these additional component(s)/reagent(s) will depend upon the particular treatment format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

The kit may be disposed in any packaging that allows the components present therein to function in accordance with the present disclosure. In certain non-limiting embodiments, the kit further comprises a sealed packaging in which the components are disposed. In certain particular (but non-limiting) embodiments, the sealed packaging is substantially impermeable to air and/or substantially impermeable to light.

In addition, the kit can further include a set of written instructions explaining how to use one or more components of the kit. A kit of this nature can be used in any of the methods described or otherwise contemplated herein.

In certain non-limiting embodiments, the kit has a shelf life of at least about six months, such as (but not limited to), at least about nine months, or at least about 12 months.

Certain non-limiting embodiments of the present disclosure are related to systems that include any of the components of the alternating electric field generating systems disclosed or otherwise contemplated herein (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with at least one of any of the compositions disclosed or otherwise contemplated herein. The systems may optionally further include one or more of any of the optional compositions disclosed or otherwise contemplated herein. The systems may optionally further include one or more devices (or one or more components of devices) utilized in one or more additional therapy steps.

It is clearly seen that a long-felt but unmet need exists in that glioblastoma cancers and other cancers are increasing in human subjects, and new effective treatments for glioblastoma (and other cancers) are needed that will combine tumor treating fields (TTFields) with a composition for treatments, wherein the composition comprises at least one inhibitor of at least one immunomodulatory cytokine. The technology herein provides, in some embodiments, a method for reducing viability of cancer cells, the method comprising the steps of: (1) applying tumor treating fields (TTFields) to the cancer cells for a period of time; and (2) administering at least one composition to the cancer cells, wherein the at least one composition comprises at least one inhibitor of at least one immunomodulatory cytokine selected from the group consisting of macrophage migration inhibitory factor (MIF), MHC class I chain-related polypeptide A (MICA), and/or MHC class I chain-related polypeptide B (MICB).

According to some aspects and detailed embodiments, the technology disclosed herein can be detailed by combining any of the following aspects with any other feature, embodiment, or example disclosed herein:

Aspect 1: A method for reducing viability of cancer cells, the method comprising: applying tumor treating fields (TTFields) to the cancer cells for a duration of at least 12 hours per day, wherein the TTFields are alternating electric fields with a frequency between about 100 kHz and about 300 kHz, preferably about 200 kHz, and a field intensity between about 1 V/cm and about 5 V/cm, preferably between about 2 V/cm and about 4 V/cm; and administering a composition to the cancer cells, wherein the composition comprises an inhibitor of an immunomodulatory cytokine selected from the group consisting of macrophage migration inhibitory factor (MIF), MHC class I chain-related polypeptide A (MICA), and MHC class I chain-related polypeptide B (MICB), and wherein the inhibitor is an antibody, preferably a monoclonal antibody or single-chain variable fragment (scFv) that is humanized or fully human, an antigen-binding fragment thereof, or a small molecule inhibitor with a molecular weight less than about 1000 daltons.

Aspect 2: The method of aspect 1, wherein the inhibitor is an inhibitor of MICB that enhances anti-tumor immunity by preventing tumor cells from shedding MICA/B, which inhibits NK cell activation, and wherein the inhibitor of MICB upregulates secreted levels of MICA/B and inhibits shedding of MICA/B to provide increased cancer treatment efficacy; or wherein the inhibitor is the MICA/MICB antibody CLN-619 that functions by restoring MICA/B expression on the surface of tumor cells, enhancing the interaction between MICA and NKG2D, and inducing antibody-dependent cellular toxicity (ADCC) to promote anti-tumor activity via NKG2D-expressing NK and T cells by inhibiting MICA/B shedding; or wherein the inhibitor is the MICA/MICB antibody DM919.

Aspect 3: The method of aspect 2, wherein the tumor treating fields (TTFields) are applied at the frequency of about 200 kHz for optimal efficacy in reducing cancer cell viability while minimizing off-target effects on healthy cells and tissues.

Aspect 4: The method of aspect 1, wherein the tumor treating fields (TTFields) have the intensity between about 2 V/cm and about 4 V/cm to provide effective cancer cell disruption while minimizing adverse effects on healthy tissues and being well-tolerated by subjects undergoing treatment.

Aspect 5: The method of aspect 1, wherein the duration of TTFields application is at least about 12 hours per day and up to about 24 hours per day to maximize the cumulative effect on cancer cells while allowing for breaks in treatment to manage any potential side effects.

Aspect 6: The method of aspect 1, wherein the inhibitor is the monoclonal antibody or the single-chain variable fragment (scFv) to provide high specificity and affinity for the target immunomodulatory cytokine, enabling effective inhibition at lower doses compared to less specific inhibitors.

Aspect 7: The method of aspect 6, wherein the antibody or antigen-binding fragment thereof is humanized or fully human to reduce immunogenicity and improve tolerability in human subjects, allowing for repeated administration over longer treatment periods.

Aspect 8: The method of aspect 1, wherein the inhibitor is the small molecule inhibitor with a molecular weight less than about 1000 daltons to enable oral administration and better penetration into solid tumor tissues, which can be more difficult to access with larger molecule inhibitors.

Aspect 9: The method of aspect 1, wherein the inhibitor is administered intravenously, orally, subcutaneously, intramuscularly, topically, transdermally, intrathecally, or via inhalation, depending on the inhibitor type and target location, to achieve optimal delivery and bioavailability while minimizing systemic exposure and off-target effects.

Aspect 10: The method of aspect 1, wherein the cancer cells are selected from the group consisting of brain cancer cells, breast cancer cells, lung cancer cells, colon cancer cells, liver cancer cells, stomach cancer cells, ovarian cancer cells, skin cancer cells, pancreatic cancer cells, and combinations thereof, demonstrating broad applicability of the method to a wide range of cancer types.

Aspect 11: The method of aspect 1, wherein the cancer cells are glioblastoma multiforme (GBM) cells, and wherein the TTFields are applied directly to the brain of a subject via transducer arrays placed on the scalp to treat this aggressive and difficult-to-treat type of brain cancer, which has limited treatment options and poor prognosis.

Aspect 12: A method for treating a subject having a cancer, the method comprising: applying tumor treating fields (TTFields) to a region of the subject containing cancer cells for a duration of at least 18 hours per day, wherein the TTFields are alternating electric fields with a frequency between about 100 kHz and about 300 kHz and a field intensity between about 1 V/cm and about 5 V/cm, preferably between about 2 V/cm and about 4 V/cm; and administering a composition to the subject, wherein the composition comprises an inhibitor of an immunomodulatory cytokine selected from the group consisting of macrophage migration inhibitory factor (MIF), MHC class I chain-related polypeptide A (MICA), and MHC class I chain-related polypeptide B (MICB), and wherein the inhibitor is an antibody, preferably a monoclonal antibody or single-chain variable fragment (scFv), an antigen-binding fragment thereof, or a small molecule inhibitor with a molecular weight less than about 1000 daltons.

Aspect 13: The method of aspect 12, wherein the inhibitor is an inhibitor of MICB that enhances anti-tumor immunity by preventing tumor cells from shedding MICA/B, which inhibits NK cell activation, and wherein the inhibitor of MICB upregulates secreted levels of MICA/B and inhibits shedding of MICA/B to provide increased cancer treatment efficacy; or wherein the inhibitor is the MICA/MICB antibody CLN-619 that functions by restoring MICA/B expression on the surface of tumor cells, enhancing the interaction between MICA and NKG2D, and inducing antibody-dependent cellular toxicity (ADCC) to promote anti-tumor activity via NKG2D-expressing NK and T cells by inhibiting MICA/B shedding; or wherein the inhibitor is the MICA/MICB antibody DM919.

Aspect 14: The method of aspect 13, wherein the tumor treating fields (TTFields) have the intensity between about 2 V/cm and about 4 V/cm to balance effectiveness with safety and tolerability in human subjects, as higher intensities may cause discomfort or adverse effects.

Aspect 15: The method of aspect 12, wherein the duration of TTFields application is at least about 18 hours per day and up to about 24 hours per day to provide sustained and continuous disruption of cancer cell division while still allowing for periodic breaks to manage potential side effects and maintain quality of life.

Aspect 16: The method of aspect 12, wherein the inhibitor is the monoclonal antibody or the single-chain variable fragment (scFv) to provide high specificity and affinity for the target immunomodulatory cytokine while minimizing off-target effects, which is particularly important when administering systemically to a subject.

Aspect 17: The method of aspect 12, wherein the inhibitor is the small molecule inhibitor with a molecular weight less than about 1000 daltons and is administered orally for ease of administration and better bioavailability, particularly for outpatient treatment, as frequent clinic visits for intravenous administration can be burdensome for patients.

Aspect 18: The method of aspect 12, wherein the cancer is selected from the group consisting of brain cancer, breast cancer, lung cancer, colon cancer, liver cancer, stomach cancer, ovarian cancer, skin cancer, pancreatic cancer, and combinations thereof, demonstrating the potential for broad clinical application to many of the most common and deadly types of cancer.

Aspect 19: The method of aspect 12, wherein the cancer is glioblastoma multiforme (GBM), and wherein the TTFields are applied directly to the brain of the subject to provide localized treatment of this difficult-to-treat brain cancer, potentially improving outcomes for patients with an otherwise very poor prognosis.

Aspect 20: The method of aspect 12, wherein administering the composition reduces a size or growth rate of a tumor comprising the cancer cells in the subject by at least about 25%, preferably at least about 50%, compared to a subject not administered the composition, demonstrating significant clinical benefit of the combination therapy and the potential to meaningfully improve progression-free and overall survival for cancer patients.

Aspect 21: A method for reducing viability of cancer cells in a subject, the method comprising: applying tumor treating fields (TTFields) to the cancer cells using a TTFields device configured to generate alternating electric fields with a frequency between 100 kHz and 300 kHz and a field intensity between 1 V/cm and 10 V/cm, wherein the TTFields is applied for a period of time ranging from 1 hour to 24 hours per day, and wherein the TTFields device comprises a plurality of insulated electrodes arranged in a predetermined geometric configuration to deliver the alternating electric fields to a target region containing the cancer cells; and administering at least one composition to the cancer cells, wherein the at least one composition comprises at least one inhibitor of at least one immunomodulatory cytokine selected from the group consisting of macrophage migration inhibitory factor (MIF), MHC class I chain-related polypeptide A (MICA), and MHC class I chain-related polypeptide B (MICB), wherein the at least one inhibitor is selected from the group consisting of small molecule inhibitors, antibodies, aptamers, siRNAs, shRNAs, miRNAs, ribozymes, and antisense oligonucleotides, and wherein the at least one composition further comprises at least one pharmaceutically acceptable carrier, diluent, or excipient.

Aspect 22: The method of aspect 21, wherein applying tumor treating fields (TTFields) to the cancer cells is performed for a period of time ranging from 4 hours to 18 hours per day, and wherein the TTFields device is configured to deliver the alternating electric fields in multiple sequential sessions, each session lasting between 1 hour and 6 hours.

Aspect 23: The method of aspect 21, wherein the at least one inhibitor is an inhibitor of macrophage migration inhibitory factor (MIF), wherein the inhibitor is selected from the group consisting of 4-iodo-6-phenylpyrimidine (4-IPP), (S,R)-3-(4-hydroxyphenyl)-4,5-dihydro-5-isoxazole acetic acid (ISO-1), and anti-MIF antibodies, and wherein the inhibitor is administered at a dose ranging from 0.01 mg/kg to 100 mg/kg body weight of the subject.

Aspect 24: The method of aspect 21, wherein the at least one inhibitor is an inhibitor of MHC class I chain-related polypeptide A (MICA), wherein the inhibitor is selected from the group consisting of anti-MICA antibodies, MICA-specific aptamers, and small molecule inhibitors of MICA shedding, and wherein the inhibitor is administered at a dose ranging from 0.01 mg/kg to 100 mg/kg body weight of the subject.

Aspect 25: The method of aspect 21, wherein the at least one inhibitor is an inhibitor of MHC class I chain-related polypeptide B (MICB), wherein the inhibitor is selected from the group consisting of anti-MICB antibodies, MICB-specific aptamers, and small molecule inhibitors of MICB shedding, and wherein the inhibitor is administered at a dose ranging from 0.01 mg/kg to 100 mg/kg body weight of the subject.

Aspect 26: The method of aspect 21, wherein the at least one composition comprises a plurality of inhibitors, each inhibitor targeting a different immunomodulatory cytokine selected from the group consisting of macrophage migration inhibitory factor (MIF), MHC class I chain-related polypeptide A (MICA), and MHC class I chain-related polypeptide B (MICB), wherein the plurality of inhibitors are formulated in a single composition or in separate compositions for co-administration, and wherein each inhibitor is administered at a dose ranging from 0.01 mg/kg to 100 mg/kg body weight of the subject.

Aspect 27: The method of aspect 21, wherein the cancer cells are selected from the group consisting of glioblastoma multiforme cells, medulloblastoma cells, breast adenocarcinoma cells, non-small cell lung cancer cells, small cell lung cancer cells, colon adenocarcinoma cells, hepatocellular carcinoma cells, pancreatic ductal adenocarcinoma cells, and ovarian carcinoma cells, and wherein the cancer cells are located in a primary tumor site, a metastatic site, or a combination thereof.

Aspect 28: The method of aspect 21, wherein administering the at least one composition is performed simultaneously with applying the tumor treating fields (TTFields), wherein the at least one composition is administered continuously or intermittently during the application of TTFields, and wherein the at least one composition is administered at a frequency ranging from once daily to once weekly.

Aspect 29: The method of aspect 21, wherein administering the at least one composition is performed sequentially with applying the tumor treating fields (TTFields), wherein the at least one composition is administered before, during, or after the application of TTFields, and wherein the at least one composition is administered at a frequency ranging from once daily to once weekly for a period of time ranging from 1 week to 52 weeks.

Aspect 30: The method of aspect 21, wherein the at least one composition is administered orally, intravenously, intraperitoneally, intramuscularly, subcutaneously, intranasally, or transdermally, wherein the at least one composition is formulated as a solution, suspension, emulsion, tablet, pill, capsule, sustained release formulation, or repository injection, and wherein the at least one composition is administered at a dose ranging from 0.01 mg to 1000 mg per administration.

Aspect 31: A system for reducing viability of cancer cells in a subject, the system comprising: a tumor treating fields (TTFields) device configured to apply alternating electric fields with a frequency between 100 kHz and 300 kHz and a field intensity between 1 V/cm and 10 V/cm to the cancer cells for a period of time ranging from 1 hour to 24 hours per day, wherein the TTFields device comprises a plurality of insulated electrodes arranged in a predetermined geometric configuration to deliver the alternating electric fields to a target region containing the cancer cells, and wherein the TTFields device further comprises a power source, a signal generator, and a control unit for adjusting the frequency, intensity, and duration of the alternating electric fields; and a composition comprising at least one inhibitor of at least one immunomodulatory cytokine selected from the group consisting of macrophage migration inhibitory factor (MIF), MHC class I chain-related polypeptide A (MICA), and MHC class I chain-related polypeptide B (MICB), wherein the at least one inhibitor is selected from the group consisting of small molecule inhibitors, antibodies, aptamers, siRNAs, shRNAs, miRNAs, ribozymes, and antisense oligonucleotides, wherein the composition further comprises at least one pharmaceutically acceptable carrier, diluent, or excipient, and wherein the composition is administered to the cancer cells.

Aspect 32: The system of aspect 31, wherein the TTFields device is configured to apply TTFields to the cancer cells for a period of time ranging from 4 hours to 18 hours per day, and wherein the TTFields device is programmed to deliver the alternating electric fields in multiple sequential sessions, each session lasting between 1 hour and 6 hours.

Aspect 33: The system of aspect 31, wherein the at least one inhibitor is an inhibitor of macrophage migration inhibitory factor (MIF), wherein the inhibitor is selected from the group consisting of 4-iodo-6-phenylpyrimidine (4-IPP), (S,R)-3-(4-hydroxyphenyl)-4,5-dihydro-5-isoxazole acetic acid (ISO-1), and anti-MIF antibodies, and wherein the inhibitor is formulated at a concentration ranging from 0.01 mg/mL to 100 mg/mL.

Aspect 34: The system of aspect 31, wherein the at least one inhibitor is an inhibitor of MHC class I chain-related polypeptide A (MICA), wherein the inhibitor is selected from the group consisting of anti-MICA antibodies, MICA-specific aptamers, and small molecule inhibitors of MICA shedding, and wherein the inhibitor is formulated at a concentration ranging from 0.01 mg/mL to 100 mg/mL.

Aspect 35: The system of aspect 31, wherein the at least one inhibitor is an inhibitor of MHC class I chain-related polypeptide B (MICB), wherein the inhibitor is selected from the group consisting of anti-MICB antibodies, MICB-specific aptamers, and small molecule inhibitors of MICB shedding, and wherein the inhibitor is formulated at a concentration ranging from 0.01 mg/mL to 100 mg/mL.

Aspect 36: The system of aspect 31, wherein the composition comprises a plurality of inhibitors, each inhibitor targeting a different immunomodulatory cytokine selected from the group consisting of macrophage migration inhibitory factor (MIF), MHC class I chain-related polypeptide A (MICA), and MHC class I chain-related polypeptide B (MICB), wherein the plurality of inhibitors are formulated in a single composition or in separate compositions for co-administration, and wherein each inhibitor is formulated at a concentration ranging from 0.01 mg/mL to 100 mg/mL.

Aspect 37: The system of aspect 31, wherein the cancer cells are selected from the group consisting of glioblastoma multiforme cells, medulloblastoma cells, breast adenocarcinoma cells, non-small cell lung cancer cells, small cell lung cancer cells, colon adenocarcinoma cells, hepatocellular carcinoma cells, pancreatic ductal adenocarcinoma cells, and ovarian carcinoma cells, and wherein the cancer cells are located in a primary tumor site, a metastatic site, or a combination thereof.

Aspect 38: The system of aspect 31, wherein the composition is administered simultaneously with the application of the TTF, wherein the composition is administered continuously or intermittently during the application of TTF, and wherein the composition is administered using an infusion pump or a controlled release device.

Aspect 39: The system of aspect 31, wherein the composition is administered sequentially with the application of the TTF, wherein the composition is administered before, during, or after the application of TTF, and wherein the composition is administered using an infusion pump or a controlled release device for a period of time ranging from 1 week to 52 weeks.

Aspect 40: The system of aspect 31, wherein the composition is formulated for oral, intravenous, intraperitoneal, intramuscular, subcutaneous, intranasal, or transdermal administration, wherein the composition is formulated as a solution, suspension, emulsion, tablet, pill, capsule, sustained release formulation, or repository injection, and wherein the composition is formulated to deliver a dose ranging from 0.01 mg to 1000 mg per administration.

According to some aspects, the invention provides a method and system for reducing the viability of cancer cells in a subject by applying tumor treating fields (TTFields) using a device configured to generate alternating electric fields with specific frequency and intensity parameters. The method includes administering a composition comprising inhibitors of immunomodulatory cytokines, such as macrophage migration inhibitory factor (MIF), MHC class I chain-related polypeptide A (MICA), and MHC class I chain-related polypeptide B (MICB). The inhibitors can be small molecule inhibitors, antibodies, aptamers, siRNAs, shRNAs, miRNAs, ribozymes, or antisense oligonucleotides. The TTFields is applied for a specified duration each day, and the composition can be administered in various forms and routes, either simultaneously or sequentially with the TTFields application. The system includes a TTFields device and the composition for administration to the cancer cells.

Cancer remains one of the leading causes of mortality worldwide, with millions of new cases diagnosed each year. Traditional cancer treatments, such as chemotherapy, radiation, and surgery, often come with significant side effects and may not be effective for all types of cancer. As a result, there is a continuous need for innovative approaches that can target cancer cells more effectively while minimizing harm to healthy cells.

One area of research that has gained attention is the use of tumor treating fields (TTFields), which are low-intensity, alternating electric fields that disrupt cancer cell division and inhibit tumor growth. TTFields has shown promise in treating certain types of cancer, such as glioblastoma, by interfering with the mitotic process of cancer cells.

In addition to TTFields, the role of the immune system in cancer progression and treatment has become a focal point of research. Immunomodulatory cytokines, which are proteins that regulate immune responses, can influence the tumor microenvironment and affect cancer cell survival. Targeting specific cytokines that promote cancer cell viability presents a potential therapeutic strategy.

However, the complexity of the immune system and the redundancy of cytokine functions pose challenges in developing effective treatments. Therefore, there is a growing interest in exploring combination therapies that integrate different modalities to enhance treatment efficacy and overcome resistance mechanisms in cancer cells.

The technology can be inter-combined with machine learning, advanced monitoring, and clinical outcomes as is discussed below.

Examples of Tumor Treating Fields Inducing the Integrated Stress Response, Alter the Transcriptional Signatures of Cellular Metabolism, and Modulate Immune-Related Cytokines Dependent and Independent of P53LE of Cellular Senescence in Aging and Age-Associated Diseases

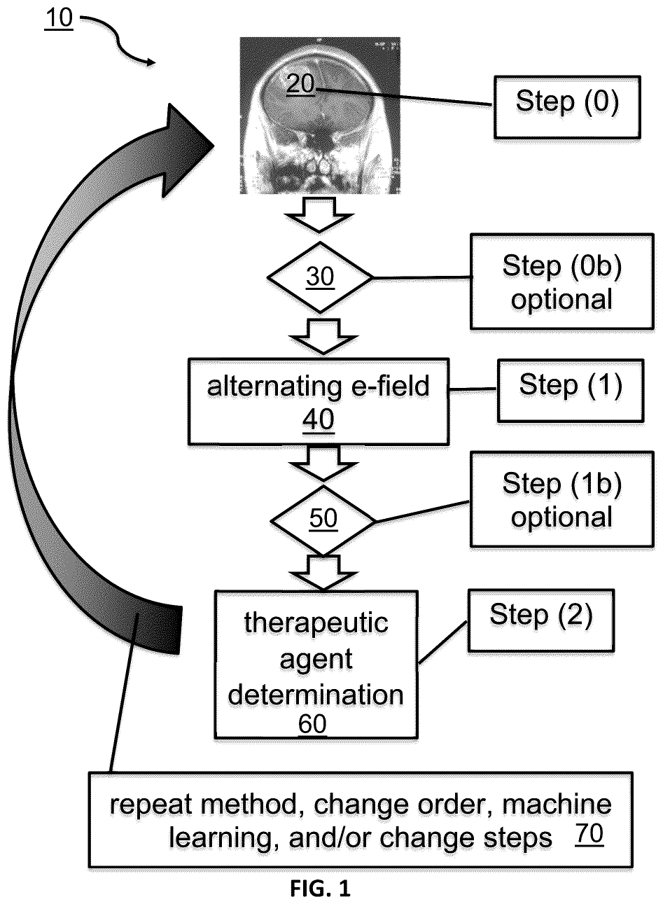

Turning now to more details optionally inter-combined with the technology disclosed above, also disclosed herein are biomarkers, cytokines, synergies and rationales for therapeutic combinations exploiting molecular alterations induced upon cancer cells by TTFields. As an introduction to details, FIG. 1 is an example of a method 10 for inducing cytostatic and/or cytotoxic effects on a cancer cell 20 (or for treating a cancer). The cancer cell 20 (or cells) can be in a subject who has (or is suspected of having) a cancer (e.g., a glioblastoma 20), or the cell 20 can be, for example, in vitro. The method 10 is illustrated comprising the steps of Step (1) (e.g., at 40) applying an alternating electric field or tumor treating fields (TTFields) to the cancer; and Step (2) contacting the cancer with a therapeutic agent, before, after, or during an execution of step (1). It should be understood that the block arrows in FIG. 1 are illustrative, and as discussed below, the method 10, can be executed in different order(s), repeated, executed backwards, or configured as a method for treating a subject with cancer. The optional steps are discussed in more detail, and the benefits of the method 10 will next be discussed in more detail.

In this example, turning to and examining FIG. 1, a method 10 of treating a subject for cancer is described. The cancer 20 or cancer cell(s) can be within a subject. In FIG. 1, the cancer 20 is depicted as (glioblastoma) in a head image, but the cancer (or cancer cells) can be at any location in a subject, any location or in vitro. Step (0) can be, in some embodiments, described as obtaining a subject in need of a cancer treatment, suspected of having a cancer, or in need of a diagnosis for cancer. In some embodiments, 20 represents a cancer cell to be induced upon a cytostatic and/or cytotoxic effect. In this example, step (0) is the step of providing or obtaining a cancer cell.

Step (0b) is an optional step at 30, which represents measuring a transcriptome and/or a secretome of the cell (or cancer 20) before execution of step (1), which is an application of TTFields (or an alternating electric field) at 40 to the cancer or cancer cell. The measuring at Step (0b) can provide, according to some aspects, pre-treatment data. The pre-treatment data can be evaluated by, for example, machine learning, comparison to genomic/proteomic databases, an AI image analysis, comparison to post-treatment data, a skilled healthcare evaluation, or by any means known.

Step (1b) is an optional step at 50, which represents measuring a transcriptome and/or a secretome of the cell (or cancer 20) after or during execution of step (1), which is an application of TTFields (or an alternating electric field) at 40 to the cancer or cancer cell. The measuring at Step (1b) can provide, according to some aspects, post-treatment data. The post-treatment data can be evaluated by, for example, machine learning, comparison to genomic/proteomic databases, an AI image analysis, comparison to pre-treatment data, skilled healthcare evaluation, or by any means known.

In some embodiments, the technology disclosed herein provides a method for determining a therapeutic agent utilized in step (2) at 60 and/or a dose or a dose regime of the agent. Such a determination can be accomplished by repeating the method, changing an order of steps, machine learning, or interpretation by a healthcare provider at 70.

To draw another example picture of an embodiment illustrated in FIG. 1, in a highly effective embodiment, the invention can be described as a method for inducing a cytostatic and/or cytotoxic effect on a cancer cell 10. The method can comprise the steps of: 40 step (1) applying an alternating electric field or tumor treating fields (TTFields) to the cancer cell 20; step (1b) measuring (at 50) one or more of: an alteration in transcriptional signatures of the cancer cell's cellular metabolism, a modulation in the cancer cell's immune-related cytokines dependent and/or independent of P53, and/or a modulation of the integrated stress response (ISR) either of the cancer cell or surrounding the cancer cell; and step (2) selecting a therapeutic agent 60 using the measuring in step (1b) and contacting the cancer cell with said therapeutic agent. In this example, step (1b) can be entirely written as: step (1b) measuring 50 a transcriptome and/or a secretome of the cancer cell after or during execution of step (1), and the measuring is operative at 60 to provide an indicative data for selecting a therapeutic agent to exploit an alteration of the cancer cell by the TTFields (or alternating electric field).

In another embodiment, a method for inducing a synergistic cytostatic and/or cytotoxic effect on a cancer cell is disclosed herein, the method comprising the steps of: (1) applying an alternating electric field or tumor treating fields (TTFields) to the cancer cell; (1b) measuring one or more of: an alteration in transcriptional signatures of the cancer cell's cellular metabolism, a modulation in the cancer cell's immune-related cytokines dependent and/or independent of P53, and/or a modulation of the integrated stress response (ISR) either of the cancer cell or surrounding the cancer cell; and (2) selecting a therapeutic agent using the measuring from step (1b) and contacting the cancer cell with said therapeutic agent; whereby a synergistic effect occurs such that the cytostatic and/or cytotoxic effect on the cancer cell is greater than compared to the cytostatic and/or cytotoxic effect (under the exact same conditions) that occurs from only the execution of step (1).

FIG. 1 is, in some embodiments, a method of reducing viability of cancer cells 10 comprising the steps of: step (1) applying an alternating electric field (TTFields) to the cancer cells, and step (2) contacting the cancer cells with a therapeutic agent; whereby the efficacy of the TTFields is increased compared to the application of TTFields without contacting the cancer cells with the therapeutic agent.

In some embodiments, the technology provides a method for inducing cytostatic and/or cytotoxic effects on a cancer cell, the method comprising the steps of: step (1) applying an alternating electric field or tumor treating fields (TTFields) to the cancer cell; and step (2) contacting the cancer cell with a therapeutic agent, before, after, and/or during execution of step (1). According to some aspects, the method can further comprise the step of: step (1b) measuring a transcriptome and/or a secretome of the cell after or during execution of step (1).

In some embodiments, the method can further comprise the step of: step (0b) measuring a transcriptome and/or a secretome of the cell before execution of step (1).

In the above described embodiments, the method can be wherein step (1b) is performed after execution of step (1) and one or more changes induced in the transcriptome and/or secretome of the cancer cell after the application of the TTFields is determined via comparing a data measured before execution of step (1) (i.e., a data 0b or pre-treatment data) and a data measured after execution of step (1) (i.e., a data 1b or post-treatment data).

It is contemplated that the method can be configured as comprising steps of a method of treating a subject in need of a cancer treatment, such that that the following method steps are executed: step (0) obtaining a subject in need of a cancer treatment; step (0b) optionally measuring a transcriptome and/or a secretome of the cancer whereby a pre-treatment data is obtained; step (1) applying an alternating electric field or tumor treating fields (TTFields) to the cancer; step (1b) optionally measuring a transcriptome and/or a secretome of the cancer after or during execution of step (1), whereby a post-treatment data is obtained; and (2) contacting the cancer with a therapeutic agent, before, after, or during an execution of step (1); wherein the contacting is done by an administration of a therapeutic agent to the subject in need thereof, or an administration of a pharmaceutical formulation thereof.

In some embodiments, a power of the technology lies in the cancer cells being assaulted by one or more alternating electric fields (TTFields) and then using RNA-seq to measure effects of the changing transcriptome and/or a secretome that is intelligently measured under TTFields (Example 2, Example 3), revealing mechanisms of harm in the cancer cells and also further revealing synergistic therapeutic agents that can be utilized to further the damages done to the cancer cells by application of additional therapeutic agents or techniques. In this example, one, two, or more different frequencies of alternating electric fields can be used simultaneously or in any order. One, two, or more therapeutic agents can be used simultaneously or in any order. One, two, or more measurements of transcriptomes and/or secretomes can be used simultaneously or in any order. The intelligence of cancer experts, RNA-seq, or artificial intelligence combined with comparison of transcriptomes and/or secretomes' changes to genome databases can be used to further exploit weaknesses in the cancer cells uncovered by the application of the TTFields. The technology enables the long felt but unmet need to diminish/cure cancers by using the enablement of the discovery that TTFields changes key cytokines (see Example 2, Example 3) uncovered using RNA-seq.

As such, the spirit of the inventive concepts disclosed herein are not limited by the range of the alternating electric fields applied, for example, in any range from about 50 kHz to about 1.5 MHz. The strength of the field can be any strength in the range from about 1 V/cm (or about 0.1 V/cm) to about 20 V/cm. The session or session of TTFields applications can be varied, for example, from about 1 hour to any time within the range up to about 6 months, with sessions therein at any time durations or ranges. The inventive concepts are not limited by the therapeutic agents that can be applied, the ADCs, the linkers, chemotherapies, or small molecules. The inventive concepts are not limited by the measurements that can be done, or when, or where done. The skilled operator will discern that the inventive concepts disclosed herein can provide methods whereby the subject is treated more effectively for the cancer (i.e., the extent the cancer is stopped or reversed) by a healthcare provider, substantially as described by the method (see FIG. 1) including all the steps of (0), (0b), (1), (1b), and (2), than compared to the same treatment, of the same subject, by the same method and the same healthcare provider without any execution of any of the steps of step (0b), step (1b), and step (2). In other words, the technology herein can save lives as enabled by Example 1, Example 2, and Example 3, and the Claims.

In some embodiments, step (0b), step (1b), and step (2) (FIG. 1, Examples 1-3) demonstrate that TTFields induce the integrated stress response (ISR), alter the transcriptional signatures of cellular metabolism, and/or modulate immune-related cytokines dependent and independent of p53 on a cancer (cell). According to some aspects, the changes to the ISR, the changes to the transcriptional signatures (e.g., secretome and/or transcriptome), and/or modulation of one or more immune-related cytokines is measured and data is obtained. In some embodiments, said data is utilized to determine a therapeutic agent to contact the cancer (cell). As discussed herein, a synergistic effect is thereby uncovered, and the embodiments disclosed herein more effectively combat the cancer (cell) compared to a sole application of TTFields.

In some embodiments, the methods described above can be wherein the pre-treatment data and/or the post-treatment data is/are compared and/or utilized to determine the therapeutic agent utilized in step (2) and/or a dose or a dose regime of the agent. According to some aspects, the methods disclosed herein can be wherein the order of the execution of the steps is changed, the method is repeated or wherein the method is executed as a continuous method.

In some embodiments, the methods can be executed wherein the pre-treatment and/or post-treatment data is compared to a previously acquired data from a different subject or to a data contained in a database, wherein the comparison includes a machine learning, sending to another location, a comparison by a healthcare provider, and/or a storing for future access.

In the methods disclosed herein, the cytostatic and/or cytotoxic effects on the cancer cell can be greater than when compared to the same effects of applying TTFields (1) without contacting the cancer cell with the therapeutic agent (2) or vice versa; whereby the cytostatic and/or cytotoxic effects on the cancer cell can be greater than when compared to contacting the cancer cell with the therapeutic agent (2) without applying the TTFields (1).

In some embodiments, the cancer cell is a cell of a subject in need of a treatment thereof and the method as practiced further induces an anti-tumor immunity or an immune response against a cancer cell in the subject.

In some embodiments, the methods discussed above can be whereby execution of step (1) induces a change, upregulation, downregulation, or modulation in a transcriptome and/or a secretome of the cancer (cell) and whereby step (2) either upregulates or downregulates said change. According to some aspects, the therapeutic agent can comprise a modulator of macrophage migration inhibitory factor (MIF) immunomodulatory cytokine. In some embodiments, MIF is upregulated independently of the p53 pathway by an execution of step (1). The modulator, in some embodiments, comprises a small molecule inhibitor of MIF, an antibody inhibitor of MIF, an anti-MIF antibody, an MIF down-regulator, or an agent that targets MIF receptors. The method can be, according to some aspects, wherein the modulator comprises an anti-CD74 monoclonal antibody, Reparixin, ISO-1 (e.g., see Table 1 and Table 2), 4-IPP, Ibudilast, RTL100, BAX69, or NbE-10 (Sparkes, et al., 2018; Kok, et al., 2018).

TABLE 1
Examples (non-limiting) of Various MIF Inhibitors (Kok, et al., 2018)

| Compound | Structure | Activity, μM Reference(s) |
|---|---|---|
| Orita-13 | 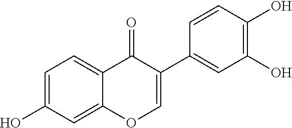 [SMILES: O=c1c(-c2ccc(O)c(O)c2)coc2cc(O)ccc12] | Kᵢ = 0.04 [39] (TA), Kᵢ = 17 [49] (TA), Kᵢ = 13-22 [40] (TA) |
| ISO-1 | 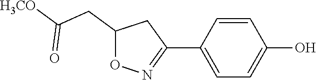 [SMILES: COC(=O)CC1CC(c2ccc(O)cc2)=NO1] | IC₅₀ = 7 [41] (TA), IC₅₀ = 24 [40] (TA); Max. 40% inhibition [42] (BA) |
| Alam-4b | 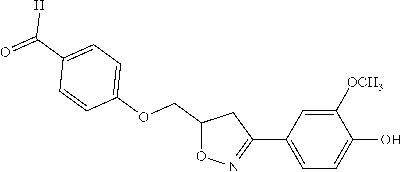 [SMILES: COc1cc(C2=NOC(COc3ccc(C=O)cc3)C2)ccc1O] | IC₅₀ = 7.3 [46] (TA) |
| ISO-66 | 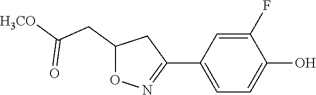 [SMILES: COC(=O)CC1CC(c2ccc(O)c(F)c2)=NO1] | IC₅₀ = 1.5 [47] (TA) |
| Jorgensen-3g | 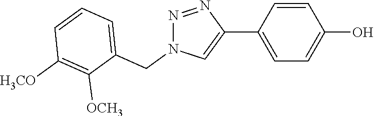 [SMILES: COc1cccc(Cn2cc(-c3ccc(O)cc3)nn2)c1OC] | IC₅₀ = 0.75 [48] (TA); IC₅₀ = 0.9 [48] (BA) |
| Jorgensen-3h | 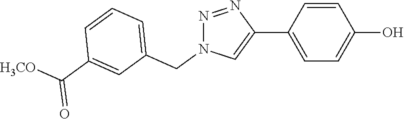 [SMILES: COC(=O)c1cccc(Cn2cc(-c3ccc(O)cc3)nn2)c1] | IC₅₀ = 1 [48] (BA) |
| Dziedzic-3bb (Cisneros-3i) | 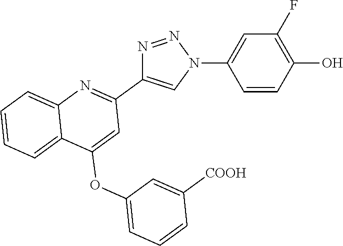 [SMILES: O=C(O)c1cccc(Oc2cc(-c3cn(-c4ccc(O)c(F)c4)nn3)nc3ccccc23)c1] | Dziedzic-3bb: Kᵢ = 0.057 [49] (TA) Cisneros-3i: Kᵢ = 0.057 [50] (TA): K_{d} = 0.071 [50] (BA) |
| Cisneros-3j | 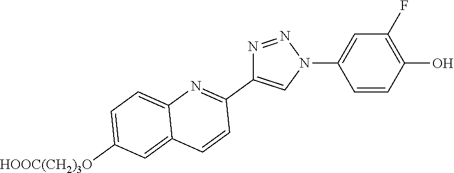 | Kᵢ = 0.034 [50] (TA); K_{d} = 0.063 [50] (BA) |

TA = tautomerase assay;
BA = binding assay MIF-CD74

TABLE 2
Examples (non-limiting) of Other MIF Inhibitors (Kok, et al., 2018)

| Compound | Structure | Activity, μM Reference(s) |
|---|---|---|
| 4-IPP | 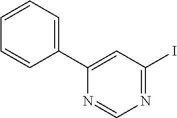 [SMILES: Ic1cc(-c2ccccc2)ncn1] | IC₅₀ = 0.2-0.5 [51] (TA) |
| BITC | 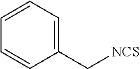 | IC₅₀ = 0.79 [53] (TA) |
| NVS-2 | 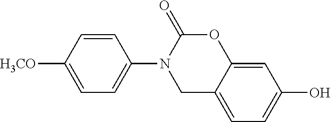 [SMILES: COc1ccc(N2Cc3ccc(O)cc3OC2=O)cc1] | IC₅₀ = 0.020 [55] (TA); Kᵢ = 0.027 [50] (TA); Kd = 0.055 [50] (BA) |
| MIF098 (Alissa-5) | 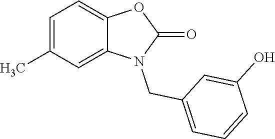 [SMILES: Cc1ccc2oc(=O)n(Cc3cccc(O)c3)c2c1] | IC₅₀ = 0.01 [56] (TA) |
| K664 1 | 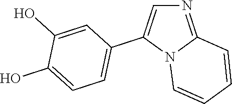 [SMILES: Oc1ccc(-c2cnc3ccccn23)cc1O] | IC₅₀ = 0.11 [59] (TA) Kᵢ = 45 [40] (TA), Kᵢ = 0.16 [58] (IA) |
| T-614 | 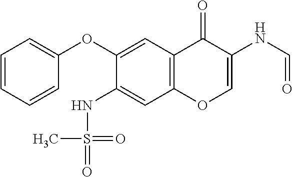 [SMILES: CS(=O)(=O)Nc1cc2occ(NC=O)c(=O)c2cc1Oc1ccccc1] | IC₅₀ = 681 [58] (TA) |
| Kok-10 | 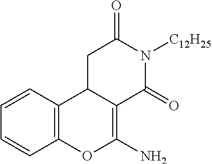 | IC₅₀ = 18 [60] (TA) |
| Kok-17 | 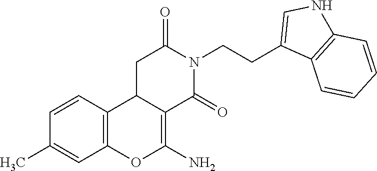 [SMILES: Cc1ccc2c(c1)OC(N)=C1C(=O)N(CCc3c[nH]c4ccccc34)C(=O)CC12] | IC₅₀ = 6.2 [60] (TA) |
| SCD-19 | 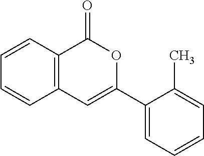 [SMILES: Cc1ccccc1-c1cc2ccccc2c(=O)o1] | 100% inhibition at concentration of 100 μM [37] (TA) |
| CPSI-2705 | 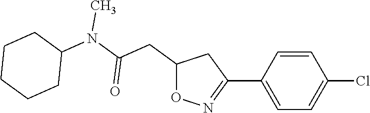 [SMILES: CN(C(=O)CC1CC(c2ccc(Cl)cc2)=NO1)C1CCCCC1] | 2-10-fold more potent than ISO-1 (35) (TA) |
| CPS1-1306 | 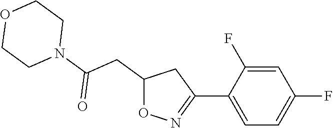 [SMILES: O=C(CC1CC(c2ccc(F)cc2F)=NO1)N1CCOCC1] | 100-fold more potent than ISO-1 [35] (TA) |

TA = tautomerase assay;
BA = binding assay MIF-CD74

In some embodiments, the therapeutic agent comprises a modulator of Interleukin 8 (IL-8) and/or whereby IL-8 is downregulated by an execution of step (1). In this example, the modulator can comprise a small molecule inhibitor of IL-8, an antibody inhibitor of IL-8, or an IL-8 down-regulator. According to some aspects, IL-8 is downregulated independently of the p53 pathway.

In some embodiments, the therapeutic agent comprises a modulator of major histocompatibility complex (MHC) class I chain related-proteins A (MICA) and/or B (MICB) and whereby MICA and/or MICB are each upregulated by an execution of step (1). For example, MICA and/or MICB can each or separately be upregulated independently of the p53 pathway. In this example, the modulator can comprise a small molecule inhibitor of MICA and/or MICB, an antibody inhibitor of MICA and/or MICB, an anti-MICA and/or MICB antibody, an MICA and/or MICB down-regulator. For example, the therapeutic agent can be configured comprising IgG1 antibody CLN-619 (Wang, et al., 2023) and/or DM919.

In some embodiments, the therapeutic agent comprises a modulator of tumor necrosis factor (TNF)-related apoptosis-inducing ligand (TRAIL)-mediated apoptosis decoy receptor DcR3 (decoy receptor 3) and/or whereby DcR3 is decreased by an execution of step (1). According to some aspects, DcR3 is decreased dependently of the p53 pathway. In this example, the modulator can comprise a small molecule inhibitor of DcR3 or an antibody inhibitor of DcR3.

In some embodiments, step (1) can induce EIF2-alpha phosphorylation, the central protein involved in activating the integrated stress response (ISR).

In some embodiments, step (1) can increase expression of surface calreticulin, an endoplasmic reticulum stress marker, which activates the ISR.

It is contemplated that the cancer cells, in various smaller embodiments, can include glioblastoma cells, mesothelioma cells, thyroid cancer cells, renal cancer cells, ovarian cancer cells, hepatocellular cancer cells, pancreatic cancer cells, lung cancer cells, breast cancer cells, colorectal cancer cells, or a combination thereof.

In some embodiments, step (1) includes a downregulation of transcripts coding for one or more enzymes involved in the Krebs cycle, fatty acid synthesis, and/or glycolysis.

According to some aspects, a method for screening a therapeutic agent for synergistic use with TTFields or method for measuring the effect(s) of an alternating electric field upon a cancer cell is disclosed herein, the method comprising the steps of: (1) applying an alternating electric field or tumor treating fields (TTFields) to the cell; (1b) measuring a change in a transcriptome and/or a secretome of one or more cancer cells from the cancer from execution of step (1); and (2) determining a therapeutic agent, a dose and/or a treatment regimen of the agent, to be administered to the subject, using the measurement from step (1b).

In some embodiments, the measuring of transcriptome and/or secretomes of cells includes RNA-seq, multiplexed cytokine profiling, observations of changes in the observable electromagnetic, an analysis by analytical chemistry techniques, PCR, cell-culture, or a combination thereof. As such, stepping towards the long felt but unmet need to cure cancers (and the increasingly tragic incidence of glioblastoma), the inventive concept is not limited by the ranges discussed herein and can include (in the claims) any endpoint subsumed within these ranges to fulfil the unmet need and to illustrate the concepts of the invention. While RNA-seq is used to measure transcriptional signatures of cellular metabolism and modulate immune-related cytokines dependent and independent of p53 herein, it is contemplated that the combination of enhanced efficacy disclosed herein can be demonstrated using other high-resolution cancer cell profiling techniques.

The methods disclosed herein, in some embodiments, can be wherein the cancer cell or cancer is in a solid tumor. The method can be executed wherein an alternating electric field is applied at a frequency in a range of from about 50 kHz to about 1.5 MHz; and the alternating electric field has a field strength of at least about 1 V/cm in at least a portion of the cancer cells or cancer. According to some aspects, the alternating electric field or TTFields include an electric field in the range from about 100 kHz to about 500 kHz or in the range from about 100 kHz to about 300 kHz.

In providing an example discussion of a Detailed Description of the methods herein, any of the aspects, embodiments, illustrations, examples, and/or features can be inter-combined with any of the details disclosed below:

Detail 1: A method for inducing an enhanced cytostatic and/or cytotoxic effect on a cancer cell, the method comprising: applying an alternating electric field or tumor treating fields (TTFields) to the cancer cell, wherein the alternating electric field or tumor treating fields (TTFields) have a frequency between about 100 kHz and about 500 kHz and an intensity between about 1 V/cm and about 10 V/cm, and wherein the alternating electric field or tumor treating fields (TTFields) are applied using insulated electrodes positioned on a skin surface proximal to the cancer cell or implanted electrodes positioned proximal to the cancer cell, and wherein the alternating electric field or tumor treating fields (TTFields) are applied for a duration between about 1 hour and about 24 hours per day and for a period between about 1 day and about 30 days; measuring one or more of an alteration in transcriptional signatures of the cancer cell's cellular metabolism, a modulation in the cancer cell's immune-related cytokines dependent and/or independent of P53, and/or a modulation of the integrated stress response (ISR) either of the cancer cell or a cell in a vicinity of the cancer cell after or during application of the alternating electric field or tumor treating fields (TTFields), wherein measuring one or more of an alteration in transcriptional signatures of the cancer cell's cellular metabolism, a modulation in the cancer cell's immune-related cytokines dependent and/or independent of P53, and/or a modulation of the integrated stress response (ISR) either of the cancer cell or a cell in a vicinity of the cancer cell is performed using a technique selected from the group consisting of RNA sequencing, microarray analysis, proteomics, metabolomics, and a combination thereof; and selecting a therapeutic agent using the one or more measurements and contacting the cancer cell with said therapeutic agent, wherein the therapeutic agent is selected from the group consisting of a chemotherapeutic agent, an immunotherapeutic agent, a targeted therapy agent, and a combination thereof, and wherein the therapeutic agent is administered systemically or locally to the cancer cell, and wherein the therapeutic agent is administered concurrently with, prior to, or after applying the alternating electric field or tumor treating fields (TTFields), and wherein the therapeutic agent is administered at a dose lower than a standard therapeutic dose or less frequently than a standard therapeutic regimen, and wherein the therapeutic agent is selected based on its ability to exploit the cellular changes induced by the alternating electric field or tumor treating fields (TTFields) to enhance the cytostatic and/or cytotoxic effect on the cancer cell; whereby the cytostatic and/or cytotoxic effect on the cancer cell is enhanced compared to the cytostatic and/or cytotoxic effect from only applying the alternating electric field or tumor treating fields (TTFields), and wherein the enhanced cytostatic and/or cytotoxic effect is an additive effect or a synergistic effect, and wherein the enhanced cytostatic and/or cytotoxic effect results in increased cancer cell death, increased cancer cell apoptosis, increased cancer cell necrosis, increased cancer cell autophagy, increased cancer cell senescence, decreased cancer cell proliferation, decreased cancer cell migration, decreased cancer cell invasion, or a combination thereof.

Detail 2: The method of detail 1, wherein measuring one or more of an alteration in transcriptional signatures of the cancer cell's cellular metabolism, a modulation in the cancer cell's immune-related cytokines dependent and/or independent of P53, and/or a modulation of the integrated stress response (ISR) either of the cancer cell or a cell in a vicinity of the cancer cell comprises measuring a transcriptome and/or a secretome of the cancer cell, wherein the transcriptome is measured using RNA sequencing or microarray analysis, and wherein the secretome is measured using proteomics or metabolomics, and wherein measuring the transcriptome and/or secretome provides a comprehensive analysis of the cellular changes induced by the alternating electric field or tumor treating fields (TTFields) at the gene expression, protein, and metabolite levels, and wherein the transcriptome analysis comprises measuring the expression levels of at least 1,000 genes, and wherein the secretome analysis comprises measuring the levels of at least 100 proteins or metabolites secreted by the cancer cell, and wherein the transcriptome and/or secretome analysis is performed at multiple time points after applying the alternating electric field or tumor treating fields (TTFields) to identify dynamic changes in gene expression, protein levels, or metabolite levels over time.

Detail 3: The method of detail 1, further comprising providing data indicative for selecting the therapeutic agent to exploit cellular changes induced by the alternating electric field or tumor treating fields (TTFields), wherein the data is obtained from measuring the transcriptome and/or secretome of the cancer cell, and wherein the data identifies specific cellular pathways, processes, or molecules altered by the alternating electric field or tumor treating fields (TTFields) that can be targeted by the therapeutic agent to enhance the cytostatic and/or cytotoxic effect on the cancer cell, and wherein the cellular pathways, processes, or molecules altered by the alternating electric field or tumor treating fields (TTFields) comprise at least one of the following: DNA damage response pathways, cell cycle regulation pathways, apoptosis pathways, cellular stress response pathways, cellular metabolism pathways, cellular signaling pathways, cellular differentiation pathways, cellular migration pathways, cellular invasion pathways, angiogenesis pathways, immune response pathways, or a combination thereof.

Detail 4: The method of detail 1, wherein the alternating electric field or tumor treating fields (TTFields) have a frequency between about 100 kHz and about 300 kHz, and wherein the frequency is selected based on the type of cancer cell to optimize the cytostatic and/or cytotoxic effect on the cancer cell, and wherein the frequency is selected based on the dielectric properties of the cancer cell, and wherein the frequency is selected to induce maximum electric field intensity within the cancer cell, and wherein the frequency is selected to induce maximum disruption of the mitotic spindle of the cancer cell during cell division.

Detail 5: The method of detail 1, wherein the alternating electric field or tumor treating fields (TTFields) have an intensity between about 1 V/cm and about 5 V/cm, and wherein the intensity is selected based on the type of cancer cell and the location of the cancer cell to minimize side effects while maintaining the cytostatic and/or cytotoxic effect on the cancer cell, and wherein the intensity is selected based on the size and shape of the cancer cell, and wherein the intensity is selected to induce maximum electric field intensity within the cancer cell while minimizing damage to surrounding healthy tissue, and wherein the intensity is selected based on the sensitivity of the cancer cell to the alternating electric field or tumor treating fields (TTFields).

Detail 6: The method of detail 1, wherein the cancer cell is a solid tumor cell, and wherein the solid tumor is selected from the group consisting of a brain tumor, a head and neck tumor, a breast tumor, a liver tumor, a lung tumor, a pancreatic tumor, a prostate tumor, a colorectal tumor, a skin tumor, and a bone tumor, and wherein the type of solid tumor is determined by histological analysis or imaging techniques such as CT, MRI, or PET scans, and wherein the histological analysis comprises examining the morphology, cellular architecture, and molecular markers of the tumor tissue, and wherein the imaging techniques comprise measuring the size, location, and metabolic activity of the tumor.

Detail 7: The method of detail 6, wherein the solid tumor is a glioblastoma multiforme (GBM) brain tumor, and wherein applying the alternating electric field or tumor treating fields (TTFields) to the GBM brain tumor comprises positioning the insulated electrodes on the scalp of a patient or implanting the electrodes in the brain of the patient proximal to the GBM brain tumor, and wherein the alternating electric field or tumor treating fields (TTFields) are applied at a frequency between about 100 kHz and about 300 kHz and an intensity between about 1 V/cm and about 5 V/cm, and wherein the alternating electric field or tumor treating fields (TTFields) are applied for a duration between about 18 hours and about 24 hours per day and for a period between about 7 days and about 30 days, and wherein the therapeutic agent is temozolomide administered orally at a dose between about 50 mg/m2/day and about 200 mg/m2/day for 5 days every 28 days.

Detail 8: The method of detail 1, wherein the therapeutic agent is a chemotherapeutic agent selected from the group consisting of temozolomide, cisplatin, carboplatin, paclitaxel, docetaxel, doxorubicin, etoposide, irinotecan, and a combination thereof, and wherein the chemotherapeutic agent is administered intravenously, orally, or directly to the tumor site, and wherein the dose and schedule of the chemotherapeutic agent is selected based on the type and stage of the cancer, the patient's age and health status, and the patient's response to previous treatments, and wherein the dose of the chemotherapeutic agent is between about 10% and about 80% of the standard therapeutic dose, and wherein the schedule of the chemotherapeutic agent is between about 50% and about 100% of the standard therapeutic schedule.

Detail 9: The method of detail 1, wherein the therapeutic agent is an immunotherapeutic agent selected from the group consisting of an immune checkpoint inhibitor, a CART-cell therapy, a cancer vaccine, and a combination thereof, and wherein the immunotherapeutic agent is administered intravenously, subcutaneously, or directly to the tumor site, and wherein the dose and schedule of the immunotherapeutic agent is selected based on the type and stage of the cancer, the patient's age and health status, and the patient's response to previous treatments, and wherein the dose of the immunotherapeutic agent is between about 10% and about 80% of the standard therapeutic dose, and wherein the schedule of the immunotherapeutic agent is between about 50% and about 100% of the standard therapeutic schedule, and wherein the immunotherapeutic agent is selected based on the expression levels of immune checkpoint proteins, tumor-associated antigens, or other immune-related molecules in the cancer cell or the tumor microenvironment.

Detail 10: The method of detail 1, wherein the therapeutic agent is a targeted therapy agent selected from the group consisting of a small molecule inhibitor, a monoclonal antibody, an antibody-drug conjugate, and a combination thereof, and wherein the targeted therapy agent is administered intravenously, orally, or directly to the tumor site, and wherein the dose and schedule of the targeted therapy agent is selected based on the type and stage of the cancer, the patient's age and health status, and the patient's response to previous treatments, and wherein the dose of the targeted therapy agent is between about 10% and about 80% of the standard therapeutic dose, and wherein the schedule of the targeted therapy agent is between about 50% and about 100% of the standard therapeutic schedule, and wherein the targeted therapy agent is selected based on the expression levels or activity of specific molecular targets in the cancer cell, such as growth factor receptors, protein kinases, transcription factors, or other oncogenic molecules.

Detail 11: The method of detail 1, wherein the enhanced cytostatic and/or cytotoxic effect is an additive effect, and wherein the additive effect is a result of the alternating electric field or tumor treating fields (TTFields) and the therapeutic agent acting independently on different cellular pathways or processes to enhance cancer cell death or inhibit cancer cell growth and proliferation, and wherein the additive effect is measured by comparing the cytostatic and/or cytotoxic effect of the combination therapy to the sum of the individual cytostatic and/or cytotoxic effects of the alternating electric field or tumor treating fields (TTFields) and the therapeutic agent alone, and wherein the additive effect is considered significant if the cytostatic and/or cytotoxic effect of the combination therapy is greater than the sum of the individual effects by at least 10%, and wherein the additive effect is optimized by adjusting the frequency, intensity, duration, and schedule of the alternating electric field or tumor treating fields (TTFields) and the dose, route of administration, and schedule of the therapeutic agent.

Detail 12: The method of detail 1, wherein the enhanced cytostatic and/or cytotoxic effect is a synergistic effect, and wherein the synergistic effect is a result of the alternating electric field or tumor treating fields (TTFields) and the therapeutic agent acting cooperatively on the same cellular pathways or processes to enhance cancer cell death or inhibit cancer cell growth and proliferation, and wherein the synergistic effect is greater than the sum of the individual effects of the alternating electric field or tumor treating fields (TTFields) and the therapeutic agent alone, and wherein the synergistic effect is measured by calculating the combination index (CI) using the Chou-Talalay method, and wherein a CI value less than 1 indicates synergy, and wherein the degree of synergy is determined by the magnitude of the CI value, and wherein the synergistic effect is optimized by adjusting the frequency, intensity, duration, and schedule of the alternating electric field or tumor treating fields (TTFields) and the dose, route of administration, and schedule of the therapeutic agent based on the CI value.

Detail 13: The method of detail 1, wherein measuring one or more of an alteration in transcriptional signatures of the cancer cell's cellular metabolism, a modulation in the cancer cell's immune-related cytokines dependent and/or independent of P53, and/or a modulation of the integrated stress response (ISR) either of the cancer cell or a cell in a vicinity of the cancer cell is performed using RNA sequencing, and wherein RNA sequencing comprises extracting RNA from the cancer cell, preparing a cDNA library, sequencing the cDNA library, and analyzing the sequencing data to identify alterations in gene expression induced by the alternating electric field or tumor treating fields (TTFields), and wherein the RNA is extracted from the cancer cell using a commercially available RNA isolation kit, and wherein the cDNA library is prepared using a commercially available cDNA synthesis kit, and wherein the cDNA library is sequenced using a high-throughput sequencing platform such as Illumina or Ion Torrent, and wherein the sequencing data is analyzed using bioinformatics software such as TopHat, Cufflinks, or DESeq2 to align the sequencing reads to a reference genome, quantify gene expression levels, and identify differentially expressed genes between treated and untreated cancer cells.

Detail 14: The method of detail 1, wherein the alternating electric field or tumor treating fields (TTFields) are applied using insulated electrodes positioned on a skin surface proximal to the cancer cell, and wherein the insulated electrodes comprise a conductive material such as hydrogel, silver, or carbon, and wherein the insulated electrodes are connected to a portable battery-powered device that generates the alternating electric field or tumor treating fields (TTFields), and wherein the portable device comprises a microprocessor, a signal generator, an amplifier, and a power source, and wherein the microprocessor controls the frequency, intensity, and duration of the alternating electric field or tumor treating fields (TTFields) based on user input or a pre-programmed treatment protocol, and wherein the signal generator produces a sinusoidal waveform at the specified frequency and amplitude, and wherein the amplifier increases the power of the signal to the specified intensity, and wherein the power source provides electrical energy to the device and is rechargeable or replaceable.

Detail 15: The method of detail 1, wherein the alternating electric field or tumor treating fields (TTFields) are applied using implanted electrodes positioned proximal to the cancer cell, and wherein the implanted electrodes comprise a biocompatible conductive material such as platinum, iridium, or titanium, and wherein the implanted electrodes are connected to a subcutaneous generator that generates the alternating electric field or tumor treating fields (TTFields), and wherein the subcutaneous generator comprises a microprocessor, a signal generator, an amplifier, and a power source, and wherein the microprocessor controls the frequency, intensity, and duration of the alternating electric field or tumor treating fields (TTFields) based on user input or a pre-programmed treatment protocol, and wherein the signal generator produces a sinusoidal waveform at the specified frequency and amplitude, and wherein the amplifier increases the power of the signal to the specified intensity, and wherein the power source provides electrical energy to the device and is rechargeable or replaceable, and wherein the subcutaneous generator is implanted under the skin of the patient and is connected to the implanted electrodes via subcutaneous wires.

Detail 16: The method of detail 1, wherein the alternating electric field or tumor treating fields (TTFields) are applied for a duration between about 18 hours and about 24 hours per day, and wherein the duration is selected based on the type of cancer cell and the patient's tolerance to the alternating electric field or tumor treating fields (TTFields) to maximize the cytostatic and/or cytotoxic effect on the cancer cell while minimizing side effects, and wherein the duration is divided into multiple sessions of between about 1 hour and about 6 hours each, and wherein the patient is allowed to take breaks between sessions to perform daily activities or to sleep, and wherein the breaks are limited to less than about 2 hours per day to maintain the efficacy of the treatment.

Detail 17: The method of detail 1, wherein the alternating electric field or tumor treating fields (TTFields) are applied for a period between about 7 days and about 30 days, and wherein the period is selected based on the type and stage of the cancer, the response of the cancer cell to the alternating electric field or tumor treating fields (TTFields), and the overall health of the patient, and wherein the period is extended if the cancer cell shows a positive response to the treatment, such as a reduction in tumor size or a decrease in cancer cell proliferation, and wherein the period is shortened if the patient experiences significant side effects or if the cancer cell shows no response to the treatment, and wherein the period is divided into multiple cycles of between about 3 days and about 7 days each, and wherein the patient is allowed to recover between cycles to minimize side effects and to maintain quality of life.

Detail 18: The method of detail 1, wherein the therapeutic agent is administered at a dose that is about 10% to about 50% lower than the standard therapeutic dose, and wherein the lower dose is selected to minimize side effects while still maintaining the cytostatic and/or cytotoxic effect on the cancer cell when combined with the alternating electric field or tumor treating fields (TTFields), and wherein the lower dose is achieved by reducing the amount of the therapeutic agent administered per dose or by reducing the frequency of administration, and wherein the lower dose is adjusted based on the patient's response to the treatment and the severity of side effects, and wherein the lower dose is increased if the cancer cell shows no response to the treatment or if the patient tolerates the treatment well, and wherein the lower dose is decreased if the patient experiences significant side effects or if the cancer cell shows a strong response to the treatment.

Detail 19: The method of detail 1, wherein the therapeutic agent is administered less frequently than the standard therapeutic regimen, and wherein the less frequent administration is selected to minimize side effects while still maintaining the cytostatic and/or cytotoxic effect on the cancer cell when combined with the alternating electric field or tumor treating fields (TTFields), and wherein the less frequent administration comprises administering the therapeutic agent every other day, twice a week, or once a week, depending on the type of therapeutic agent and the type and stage of the cancer, and wherein the frequency of administration is adjusted based on the patient's response to the treatment and the severity of side effects, and wherein the frequency of administration is increased if the cancer cell shows no response to the treatment or if the patient tolerates the treatment well, and wherein the frequency of administration is decreased if the patient experiences significant side effects or if the cancer cell shows a strong response to the treatment.

Detail 20: The method of detail 1, wherein the therapeutic agent is selected based on its ability to exploit the cellular changes induced by the alternating electric field or tumor treating fields (TTFields) to enhance the cytostatic and/or cytotoxic effect on the cancer cell, and wherein the cellular changes induced by the alternating electric field or tumor treating fields (TTFields) comprise changes in cell cycle progression, DNA repair mechanisms, apoptotic pathways, metabolic pathways, or a combination thereof, and wherein the therapeutic agent is selected to target one or more of these cellular changes to enhance the cytostatic and/or cytotoxic effect on the cancer cell, and wherein the therapeutic agent is selected based on its mechanism of action, its ability to penetrate the tumor tissue, its toxicity profile, its compatibility with the alternating electric field or tumor treating fields (TTFields), and its potential for synergy with the alternating electric field or tumor treating fields (TTFields), and wherein the therapeutic agent is selected from a database of approved and investigational cancer drugs based on its predicted efficacy and safety in combination with the alternating electric field or tumor treating fields (TTFields) using machine learning algorithms and computational modeling.

Detail 21: A method for inducing an enhanced cytostatic and/or cytotoxic effect on a cancer cell, the method comprising: applying an electric field to the cancer cell, wherein the electric field is applied using a pair of insulated electrodes connected to an electric field generator that generates the electric field at a frequency between 100 kHz and 300 kHz and at a field strength between 1 V/cm and 10 V/cm, and wherein the electric field is applied for a duration between 1 hour and 24 hours per day, and wherein the electric field is applied in a pulsed manner with a duty cycle between 50% and 95%, and wherein the pulsed electric field has a pulse width between 100 μs and 1000 μs; measuring cancer cell parameters to select a therapeutic agent, wherein measuring the cancer cell parameters comprises: obtaining a sample of the cancer cell after applying the electric field, wherein the sample is obtained by performing a biopsy of a tumor tissue containing the cancer cell; isolating RNA from the sample, wherein the RNA is isolated using a commercially available RNA isolation kit; performing transcriptome analysis on the isolated RNA to measure changes in gene expression levels compared to gene expression levels prior to applying the electric field, wherein the transcriptome analysis is performed using RNA sequencing on a next-generation sequencing platform; and identifying at least one altered metabolic pathway based on the changes in gene expression levels, wherein the at least one altered metabolic pathway is selected from the group consisting of glycolysis, tricarboxylic acid (TCA) cycle, oxidative phosphorylation, pentose phosphate pathway, fatty acid synthesis, and amino acid metabolism; selecting the therapeutic agent using the cancer cell parameters, wherein the therapeutic agent is selected to target the at least one altered metabolic pathway, and wherein the therapeutic agent is selected from the group consisting of a glycolysis inhibitor, a TCA cycle inhibitor, an oxidative phosphorylation inhibitor, a pentose phosphate pathway inhibitor, a fatty acid synthesis inhibitor, and an amino acid metabolism inhibitor; and contacting the cancer cell with the selected therapeutic agent to induce the enhanced cytostatic and/or cytotoxic effect, wherein the enhanced cytostatic and/or cytotoxic effect is greater than an effect caused by only applying the electric field, and wherein the selected therapeutic agent is administered to the cancer cell at a concentration between 0.1 nM and 100 μM for a duration between 1 hour and 72 hours, and wherein the selected therapeutic agent is administered intravenously, intratumorally, or orally.

Detail 22: The method of detail 21, wherein the electric field is an alternating electric field or tumor treating fields (TTFields), and wherein the alternating electric field is applied at a frequency that disrupts mitotic spindle formation in the cancer cell during mitosis, leading to mitotic catastrophe and subsequent cell death, and wherein the frequency of the alternating electric field is selected based on the cell size, shape, and electrical properties of the cancer cell, and wherein the frequency is adjusted in real-time based on changes in the cancer cell's morphology and dielectric properties during the course of treatment.

Detail 23: The method of detail 21, wherein measuring the cancer cell parameters comprises measuring at least one of: an alteration in transcriptional signatures of the cancer cell's cellular metabolism, wherein the alteration in transcriptional signatures includes increased expression of genes involved in glycolysis, oxidative phosphorylation, and fatty acid synthesis, and wherein the increased expression of genes is determined by comparing the gene expression levels to reference gene expression levels obtained from non-cancerous cells of the same tissue type; a modulation in the cancer cell's immune-related cytokines, wherein the modulation is dependent and/or independent of P53, and wherein the immune-related cytokines include interleukin-6 (IL-6), interleukin-8 (IL-8), and tumor necrosis factor-alpha (TNF-α), and wherein the modulation of the immune-related cytokines is measured using an enzyme-linked immunosorbent assay (ELISA) or a multiplex immunoassay; a modulation of the integrated stress response (ISR) of the cancer cell, wherein the modulation of the ISR includes increased phosphorylation of eukaryotic initiation factor 2 alpha (eIF2α) and increased expression of activating transcription factor 4 (ATF4) and C/EBP homologous protein (CHOP), and wherein the increased phosphorylation of eIF2α is measured using Western blot analysis, and wherein the increased expression of ATF4 and CHOP is measured using quantitative reverse transcription polymerase chain reaction (qRT-PCR); or a modulation of the integrated stress response (ISR) of a cell in a vicinity of the cancer cell, wherein the cell in the vicinity of the cancer cell is a stromal cell or an immune cell, and wherein the modulation of the ISR in the cell in the vicinity of the cancer cell enhances the cytotoxic effect of the selected therapeutic agent on the cancer cell, and wherein the modulation of the ISR in the cell in the vicinity of the cancer cell is measured by isolating the stromal cell or immune cell from the tumor microenvironment and performing Western blot analysis or qRT-PCR to determine the levels of phosphorylated eIF2α, ATF4, and CHOP.

Detail 24: The method of detail 23, wherein measuring the cancer cell parameters further comprises measuring at least one of a transcriptome or a secretome of the cancer cell after or during applying the electric field, wherein measuring the transcriptome involves performing RNA sequencing to determine changes in gene expression levels, and wherein measuring the secretome involves performing mass spectrometry to identify secreted proteins and metabolites, and wherein the RNA sequencing and mass spectrometry are performed using samples obtained from a culture medium in which the cancer cell is grown, and wherein the culture medium is collected at multiple time points after applying the electric field to monitor dynamic changes in the transcriptome and secretome over time.

Detail 25: The method of detail 21, wherein selecting the therapeutic agent comprises selecting the therapeutic agent to exploit an alteration in the cancer cell caused by the electric field, wherein the alteration in the cancer cell includes increased oxidative stress, increased mitochondrial dysfunction, and increased accumulation of unfolded or misfolded proteins in the endoplasmic reticulum, and wherein the selected therapeutic agent is chosen from the group consisting of a chemotherapeutic agent, a targeted therapy agent, an immunotherapy agent, a metabolic inhibitor, and combinations thereof, and wherein the chemotherapeutic agent is selected from the group consisting of doxorubicin, cisplatin, paclitaxel, and 5-fluorouracil, and wherein the targeted therapy agent is selected from the group consisting of erlotinib, gefitinib, imatinib, and trastuzumab, and wherein the immunotherapy agent is selected from the group consisting of ipilimumab, nivolumab, pembrolizumab, and atezolizumab, and wherein the metabolic inhibitor is selected from the group consisting of 2-deoxyglucose, dichloroacetate, and metformin.

In some examples, any of the methods disclosed herein are executed in part using guided machine learning and big datasets (including increasing, pooled data from more and more patients). In some embodiments, the methods are partially executed using a computing system, statistical machine learning, guided machine learning, image analysis, or artificial intelligence (AI). The methods described herein can be implemented in any suitable computing system. The computing system can be implemented as or can include a computer device that includes a combination of hardware, software, and firmware that allows the computing device to run an applications layer or otherwise perform various processing tasks. Computing devices can include without limitation personal computers, workstations, servers, laptop computers, tablet computers, mobile devices, wireless devices, smartphones, wearable devices, embedded devices, microprocessor-based devices, microcontroller-based devices, programmable consumer electronics, mini-computers, main frame computers, and the like and combinations thereof.

Processing tasks can be carried out by one or more processors. Various types of processing technology can be used including a single processor or multiple processors, a central processing unit (CPU), multicore processors, parallel processors, or distributed processors. Additional specialized processing resources such as graphics (e.g., a graphics processing unit or GPU), video, multimedia, or mathematical processing capabilities can be provided to perform certain processing tasks. Processing tasks can be implemented with computer-executable instructions, such as application programs or other program modules, executed by the computing device. Application programs and program modules can include routines, subroutines, programs, scripts, drivers, objects, components, data structures, and the like that perform particular tasks or operate on data.

Processors can include one or more logic devices, such as small-scale integrated circuits, programmable logic arrays, programmable logic devices, masked-programmed gate arrays, field programmable gate arrays (FPGAs), application specific integrated circuits (ASICs), and complex programmable logic devices (CPLDs). Logic devices can include, without limitation, arithmetic logic blocks and operators, registers, finite state machines, multiplexers, accumulators, comparators, counters, look-up tables, gates, latches, flip-flops, input and output ports, carry in and carry out ports, and parity generators, and interconnection resources for logic blocks, logic units and logic cells.

The computing device includes memory or storage, which can be accessed by a system bus or in any other manner. Memory can store control logic, instructions, and/or data. Memory can include transitory memory, such as cache memory, random access memory (RAM), static random-access memory (SRAM), main memory, dynamic random-access memory (DRAM), block random access memory (BRAM), and memristor memory cells. Memory can include storage for firmware or microcode, such as programmable read only memory (PROM) and erasable programmable read only memory (EPROM). Memory can include non-transitory or nonvolatile or persistent memory such as read only memory (ROM), one-time programmable non-volatile memory (OTPNVM), hard disk drives, optical storage devices, compact disc drives, flash drives, floppy disk drives, magnetic tape drives, memory chips, and memristor memory cells. Non-transitory memory can be provided on a removable storage device. A computer-readable medium can include any physical medium that is capable of encoding instructions and/or storing data that can be subsequently used by a processor to implement embodiments of the systems and methods described herein. Physical media can include floppy discs, optical discs, CDs, mini-CDs, DVDs, HD-DVDs, Blu-ray discs, hard drives, tape drives, flash memory, or memory chips. Any other type of tangible, non-transitory storage that can provide instructions and/or data to a processor can be used in the systems and methods described herein.

The computing device can include one or more input/output interfaces for connecting input and output devices to various other components of the computing device. Input and output devices can include, without limitation, keyboards, mice, joysticks, microphones, cameras, webcams, displays, touchscreens, monitors, scanners, speakers, and printers. Interfaces can include universal serial bus (USB) ports, serial ports, parallel ports, game ports, and the like.

The computing device can access a network over a network connection that provides the computing device with telecommunications capabilities Network connection enables the computing device to communicate and interact with any combination of remote devices, remote networks, and remote entities via a communications link. The communications link can be any type of communication link including without limitation a wired or wireless link. For example, the network connection can allow the computing device to communicate with remote devices over a network which can be a wired and/or a wireless network, and which can include any combination of intranet, local area networks (LANs), enterprise-wide networks, medium area networks, wide area networks (WANS), virtual private networks (VPNs), the Internet, cellular networks, and the like. Control logic and/or data can be transmitted to and from the computing device via the network connection. The network connection can include a modem, a network interface (such as an Ethernet card), a communication port, a PCMCIA slot and card, or the like to enable transmission to and receipt of data via the communications link. A transceiver can include one or more devices that both transmit and receive signals, whether sharing common circuitry, housing, or a circuit boards, or whether distributed over separated circuitry, housings, or circuit boards, and can include a transmitter-receiver.

The computing device can include a browser and a display that allow a user to browse and view pages or other content served by a web server over the communications link. A web server, sever, and database can be located at the same or at different locations and can be part of the same computing device, different computing devices, or distributed across a network. A data center can be located at a remote location and accessed by the computing device over a network. The computer system can include architecture distributed over one or more networks, such as, for example, a cloud computing architecture. Cloud computing includes without limitation distributed network architectures for providing, for example, software as a service (SaaS).

While example embodiments have been particularly shown and described, it will be understood by those skilled in the art that various changes in form and details may be made therein without departing from the scope of the embodiments encompassed by the appended claims. For example, other useful implementations could be achieved if steps of the disclosed techniques were performed in a different order and/or if components in the disclosed systems were combined in a different manner and/or replaced or supplemented by other components. Accordingly, other implementations are within the scope of the disclosure.

### EXAMPLES

Examples are provided herein below. However, the present disclosure is to be understood to not be limited in its application to the specific experimentation, results, and laboratory procedures disclosed herein after. Rather, the Examples are simply provided as one of various embodiments and is meant to be exemplary, not exhaustive.

In some embodiments, for example, disclosed are enhanced methods for treating subjects using tumor treating fields (TTFields) by obtaining a subject (step 0) in need of cancer treatment; measuring (step 0b) a transcriptome and/or a secretome of the cancer; applying (step 1) TTFields to the cancer; measuring (step 1b) a transcriptome and/or a secretome of the cancer after execution of step (1); and contacting the cancer with a therapeutic agent (step 2), after, or during an execution of the TTFields step (1). The subject is treated more effectively for the cancer (i.e., the extent the cancer is stopped or reversed) by a healthcare provider, substantially as described by the method including the steps of step (0b), step (1b), and step (2), than compared to the same treatment of the same subject without the execution of steps (0b), step (1b), and step (2) due to synergistic method effects. However, any of the methods disclosed herein can be inter-combined with the purpose of saving human lives, as discussed below.

### Example 1. Cytokine Data Acquisition Introduction

Tumor treating fields (TTFields) is an FDA-approved therapy for the treatment of glioblastoma and malignant pleural mesothelioma. TTFields employ alternating electric fields delivered by cutaneous transducer arrays to induce cytostatic and cytotoxic effects on solid tumors. Additionally, recent studies suggest that TTFields may enhance anti-tumor immunity. RNA-seq and multiplexed cytokine profiling was performed in HCT116 wild-type and HCT116 p53-/human colorectal cancer cells to elucidate TTFields' effects on the transcriptome and secretome. TTFields led to profound changes in the transcriptome related to metabolism, including downregulation of transcripts coding for multiple enzymes involved in the Krebs cycle, fatty acid synthesis, and glycolysis. Furthermore, though only HCT116 p53-wild type cells strongly induced the p53 pathway, both cell types' transcriptomes showed strong downregulation of cell cycle progression.

Cytokine data showed that the key tumor-promoting cytokine IL-8 was downregulated independently of p53. Upregulation of Secreted immunomodulatory cytokines MIF and MICB independently of p53 was identified. While MICB promotes NK cell activation, MIF drives tumor progression. Therefore, MICB may be one way in which TTFields enhances anti-tumor immunity; Tumor cells overcome this by shedding MICA/B and thus inhibiting NK cells activation. Since secreted levels of MICA/B were upregulated, inhibiting shedding of MICA/B by a specific antibody or decoy could increase treatment efficacy. Targeting MIF during TTFields treatment may lead to synergy by abolishing its pro-tumor effects.

A p53-dependent decrease in the TRAIL decoy receptor DcR3, which normally protects tumor cells from TRAIL-induced apoptosis, was also identified. Further mechanistic work showed that TTFields induced EIF2-alpha phosphorylation, the central protein involved in activating the integrated stress response (ISR), across multiple cancer types, leading to suppression of global protein translation. TTFields treatment also resulted in increased expression of surface calreticulin, an endoplasmic reticulum stress marker, which activates the ISR.

Future mechanistic work (e.g., Example 3) will explore the impact of identified cytokines and the ISR on anti-tumor immunity both in vitro and in vivo, as well as metabolic alterations induced by TTFields. This work identifies potential biomarkers and rationales for therapeutic combinations exploiting TTFields-induced molecular alterations.

### Example 2. Comparing Cancer Patients Treated Solely with TTFields Therapy—to Cancer Patients Treated Strategically with Therapeutic Agents and TTFields

In this prophetic Example, all patients are treated using step (1) (see FIG. 1) applying an alternating electric field or tumor treating fields (TTFields) to the cancer under carefully controlled, FDA-approved guidelines.

To simultaneously compare control and experimental cohorts, a randomized, double blind cohort of patients is treated (e.g., times/durations) solely using TTFields as described by the FDA-approved therapy for the treatment of glioblastoma (control group). A second randomized, double blind cohort of patients is treated using the exact same treatment/times but also including step (0b) (FIG. 1) measuring a transcriptome and/or a secretome of the cancer whereby a pre-treatment data is obtained; step (1b) measuring a transcriptome and/or a secretome of the cancer after or during execution of step (1), whereby a post-treatment data is obtained; and step (2) (FIG. 1) determining an optimum therapeutic agent to use and contacting the cancer (or suspected cancer) with a therapeutic agent, before, after, or during an execution of step (1) (experimental group).

After determining the therapeutic agent, the contacting of the cancer by the therapeutic agent is done by an administration of a therapeutic agent to the subject(s) in need thereof, or an administration of a suitable pharmaceutical formulation, salt, or hydrate thereof.

Importantly, it is found in this Example that the subject(s) is/are treated more effectively for the cancer (i.e., the extent the cancer is stopped, reversed, or shrank; in the experimental group) by a healthcare provider, substantially as described by the method including the steps of (0b), (1b), and (2), (FIG. 1) than compared to the same treatment, of the same subject, by the same method and the same healthcare provider without any execution of any of the steps of step (0b), step (1b), and step (2) (i.e., control group). Statistical significance is utilized to clarify the improvement(s) as needed. It is found that measuring the weaknesses of the cancers' revealed by the changes in transcriptomes and/or secretomes, revealed by/during the application of the TTFields, and further (intelligently) exploiting these weaknesses by the “double hit” of applying an intelligently selected therapeutic agent more effectively treats the cancer and hopefully, saves lives.

This Example is done first in vitro and simultaneously repeated in vitro, (see Example 2), for a variety of human cancer cells, and it is found that the transcriptomes and/or secretomes of the cancer cells reveal vulnerabilities that can be synergistically exploited by contacting the cancer cells with a therapeutic agent in a treatment regimen along with TTFields; RNA-seq is used to profile the cancer cells. A kit is subsequently designed including the most common therapeutic agent(s) to be used simultaneously with a TTFields device.

### Example 3. Investigating Transcriptomes, Cytokines and Secretomes with TTFields

RNA sequencing (“RNA-seq”) has become herein a groundbreaking revolution for transcriptome profiling, in particular, for cancer treatment. Single-molecular, single-cell and spatial transcriptome approaches have enabled accurate, individual cell resolution incorporated with spatial information. Cancer, a major malignant and heterogeneous lethal disease, remains an enormous challenge in medical research and clinical treatment. As new a vital tool, RNA seq has been utilized herein, in combination with TTFields to yield unparalleled strategic groundwork for cancer research.

Cancer therapy, including biomarker discovery and characterization of cancer heterogeneity and evolution, drug resistance, cancer immune microenvironment, immunotherapy, and cancer neoantigens needs a full scientific approach using RNA seq (Hong, et al., 2020).

TTFields is an FDA-approved therapy for the treatment of glioblastoma and malignant pleural mesothelioma. TTFields employ alternating electric fields delivered by cutaneous transducer arrays to induce cytostatic and cytotoxic effects on solid tumors. Additionally, recent studies suggest that TTFields may enhance anti-tumor immunity.

Cytokine data showed that the key tumor-promoting cytokine IL-8 was downregulated independently of p53. Upregulation of immunomodulatory cytokines MIF and MICB independently of p53 was identified. While MICB promotes NK cell activation, MIF drives tumor progression. Therefore, MICB may be one way in which TTFields enhances anti-tumor immunity; targeting MIF during TTFields treatment may lead to synergy by abolishing its pro-tumor effects.

A p53-dependent decrease in the TRAIL decoy receptor DcR3, which normally protects tumor cells from TRAIL-induced apoptosis, was also identified. Further mechanistic work showed that TTFields induced EIF2-alpha phosphorylation, the central protein involved in activating the integrated stress response (ISR), across multiple cancer types, leading to suppression of global protein translation. TTFields treatment also resulted in increased expression of surface calreticulin, an endoplasmic reticulum stress marker, which activates the ISR.

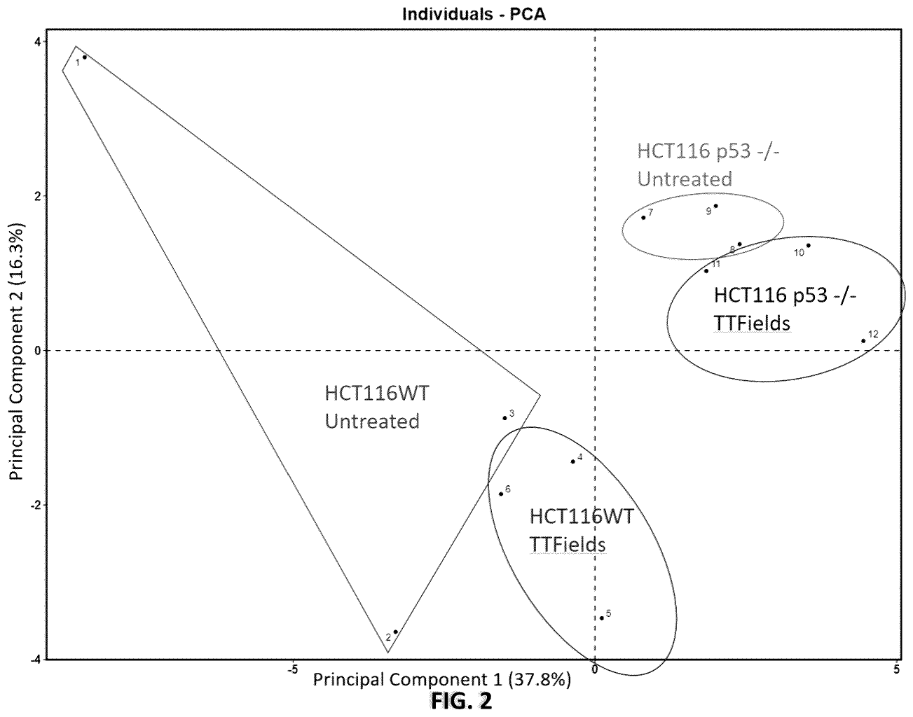

In cytokine profiling, the Experimental Design was HCT116 WT or HCT116 p53−/− were either subjected to TTFields for 24 hours (150 kHz) or left untreated for 24 hours. Then supernatants were then harvested and ran on a Luminex panel to quantify proteins in the supernatants. FIG. 2 shows a plot of cytokine profiling results. Principal component 1 is on the X-axis, with Principal component 2 on the Y-axis. Individuals' PCA are labeled.

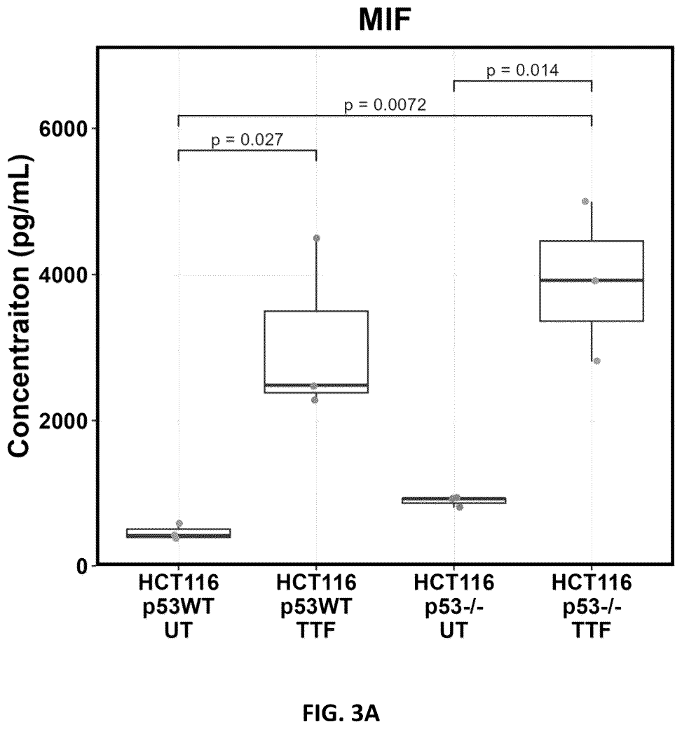

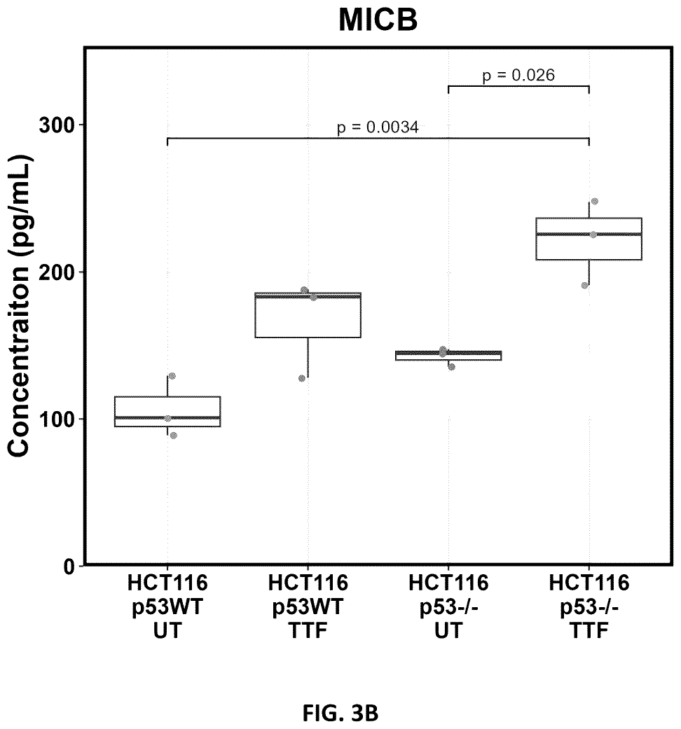

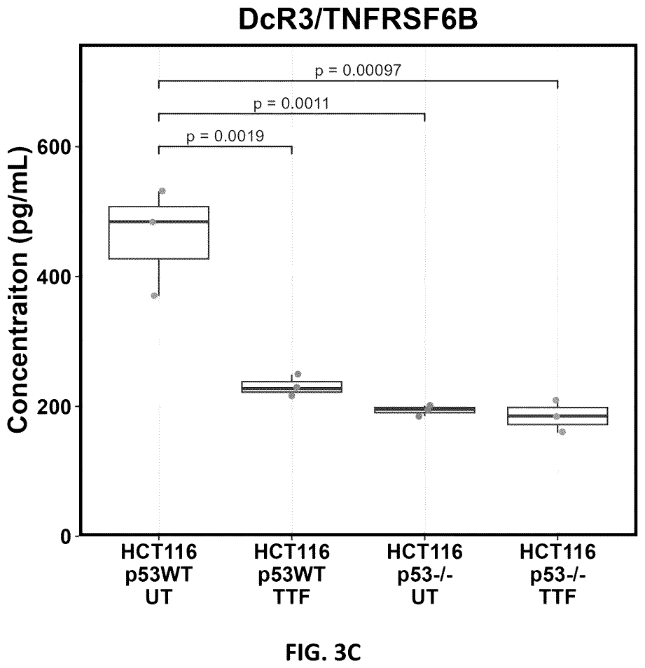

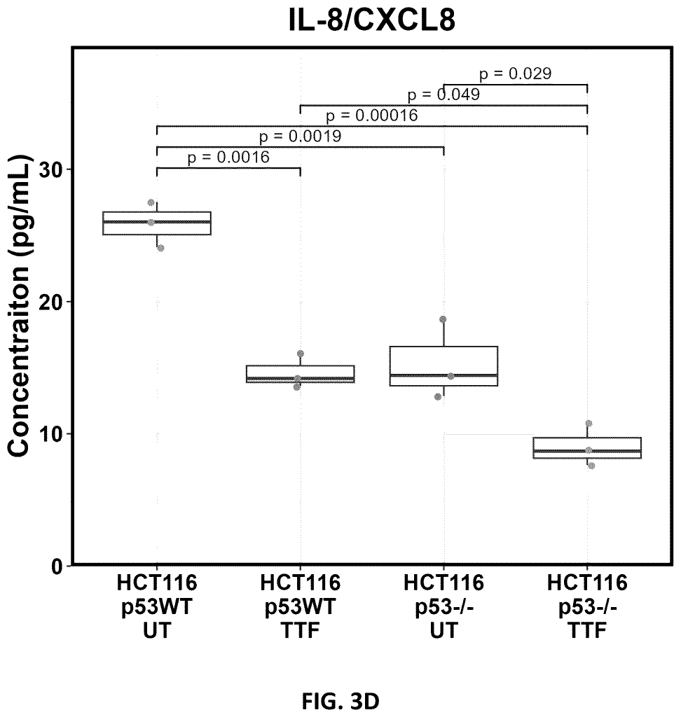

FIG. 3A and FIG. 3B show plots of example cytokines raised by TTFields treatment.

FIG. 3C and FIG. 3D show plots of example cytokines lowered by TTFields treatment. The figures represent any statistically significant differences found in cytokine profiles. The statistical method was Benjamini Hochberg method (false discovery rate) applied to account for multiple testing.

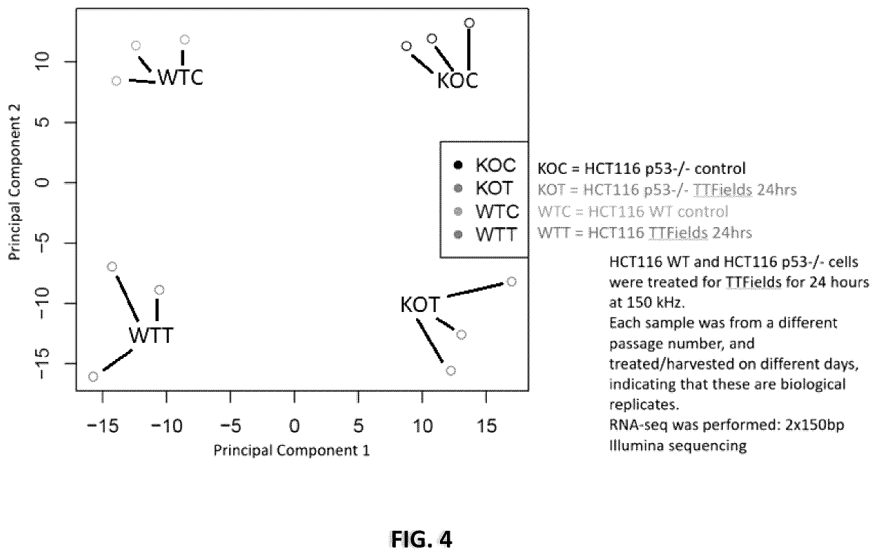

FIG. 4 shows a plot introducing RNA-seq data. HCT116 WT and HCT116 p53−/− cells were treated for TTFields for 24 hours at 150 kHz. Each sample was from a different passage number, and treated/harvested on different days, indicating that these are biological replicates. RNA-seq was performed: 2×150 bp Illumina sequencing.

The data demonstrate data used in a method for screening a therapeutic agent for a synergistic use with TTFields or a method for measuring the effect(s) of (or suspected effects of) an alternating electric field upon a cancer cell, wherein the method is comprising the steps of: (1) applying an alternating electric field or TTFields to the cell; (2) measuring a change in a transcriptome and/or a secretome of one or more cancer cells from the cancer from execution of step (1); and (3) determining a therapeutic agent, a dose and/or a treatment regimen of the agent, to be administered to the subject, using the measurement from step (2); and/or: optionally repeating the measuring and therapeutic agent so as to provide a determination of effect(s) of or synergistic use(s) of.

Future mechanistic work will explore the impact of identified cytokines and the ISR on anti-tumor immunity both in vitro and in vivo, as well as metabolic alterations induced by TTFields. This work identifies potential biomarkers and rationales for therapeutic combinations exploiting TTFields-induced molecular alterations.

Demonstrations of the technology are planned to be provided to show how the methods described herein are intuitively learned and rapidly implemented to save lives, in particular, as applied to glioblastoma.

### Non-Limiting Illustrative Embodiments of the Inventive Concept(s)

Illustrative embodiment 1. A method of reducing viability of cancer cells, the method comprising the steps of: (1) applying an alternating electric field to the cancer cells for a period of time; and (2) administering at least one composition to the cancer cells, wherein the at least one composition comprises at least one inhibitor of at least one immunomodulatory cytokine.

Illustrative embodiment 2. A method of treating cancer in a subject, the method comprising the steps of: (1) applying an alternating electric field to a target region of the subject for a period of time; and (2) administering at least one composition to the subject, wherein the at least one composition comprises at least one inhibitor of at least one immunomodulatory cytokine.

Illustrative embodiment 3. A method of reducing a volume of a tumor and/or preventing an increase of volume of the tumor, wherein the tumor is present in a body of a living subject and includes a plurality of cancer cells, the method comprising the steps of: (1) applying an alternating electric field to a target region of the subject for a period of time; and (2) administering at least one composition to the subject, wherein the at least one composition comprises at least one inhibitor of at least one immunomodulatory cytokine.

Illustrative embodiment 4. A method, comprising the steps of: (1) applying an alternating electric field to a target region of the subject for a period of time; and (2) administering at least one composition to the subject, wherein the at least one composition comprises at least one inhibitor of at least one immunomodulatory cytokine; and wherein administration of the alternating electric field increases the efficacy of the at least one composition against cancer cells in the subject when compared to the administration of the at least one composition to the subject in the absence of alternating electric field application.

Illustrative embodiment 5. The method of any of illustrative embodiments 1-4, wherein the at least one immunomodulatory cytokine is selected from the group consisting of macrophage migration inhibitory factor (MIF), MHC class I chain-related polypeptide A (MICA), MHC class I chain-related polypeptide B (MICB), and combinations thereof.

Illustrative embodiment 6. The method of any of illustrative embodiments 1-5, wherein at least one of: the alternating electric field is applied at a frequency in a range of from about 50 kHz to about 1 MHz; the alternating electric field has a field strength of at least about 1 V/cm in at least a portion of the cancer cells/target region of the subject; and the period of time that the alternating electric field is applied is at least about 50% of a 24 consecutive hour time period (i.e., at least about 12 cumulative hours of a 24 hour period).

Illustrative embodiment 7. The method of any one of illustrative embodiments 1-6, wherein the at least one composition is administered before application of the alternating electric field has begun.

Illustrative embodiment 8. The method of any one of illustrative embodiments 1-7, wherein administration of the at least one composition and initiation of application of the alternating electric field are performed wholly or partially simultaneously.

Illustrative embodiment 9. The method of any one of illustrative embodiments 1-8, wherein the at least one composition is administered after application of the alternating electric field has begun.

Illustrative embodiment 10. The method of illustrative embodiment 9, wherein the at least one composition is administered before the period of time that the alternating electric field is applied has elapsed.

Illustrative embodiment 11. The method of illustrative embodiment 9, wherein the at least one composition is administered after the period of time has elapsed.

Illustrative embodiment 12. The method of any one of illustrative embodiments 1-11, wherein one or more of the steps is repeated one or more times.

Illustrative embodiment 13. The method of any one of illustrative embodiments 1-12, wherein the cancer cells/cancer/tumor is in the form of at least one solid tumor.

Illustrative embodiment 14. The method of any one of illustrative embodiments 1-13, wherein the cancer/cancer cells is/are selected from the group consisting of hepatocellular carcinoma/carcinoma cells, glioblastoma/glioblastoma cells, pleural mesothelioma/mesothelioma cells, differentiated thyroid cancer/cancer cells, advanced renal cell carcinoma/carcinoma cells, ovarian cancer/cancer cells, pancreatic cancer/cancer cells, lung cancer/cancer cells, breast cancer/cancer cells, and combinations thereof.

Illustrative embodiment 15. The method of any one of illustrative embodiments 1-14, wherein at least a portion of the cancer cells or at least a portion of the cancer in the subject is resistant to treatment with an immunomodulatory cytokine inhibitor alone.

Illustrative embodiment 16. The method of any of illustrative embodiments 1-15, wherein the at least one immunomodulatory cytokine inhibitor comprises a small molecule inhibitor of at least one of MICA and MICB.

Illustrative embodiment 17. The method of any of illustrative embodiments 1-16, wherein the at least one immunomodulatory cytokine inhibitor comprises an antibody against at least one of MICA and MICB.

Illustrative embodiment 18. The method of illustrative embodiment 17, wherein the anti-MICA/MICB antibody comprises one or more of CLN-619 and DM919.

Illustrative embodiment 19. The method of any one of illustrative embodiments 1-18, wherein the at least one immunomodulatory cytokine inhibitor comprises a small molecule inhibitor of MIF.

Illustrative embodiment 20. The method of illustrative embodiment 19, wherein the small molecule inhibitor of MIF is selected from the group consisting of ISO-1, 4-IPP, Ibudilast, and combinations thereof.

Illustrative embodiment 21. The method of any one of illustrative embodiments 1-20, wherein the at least one immunomodulatory cytokine inhibitor comprises an anti-MIF antibody.

Illustrative embodiment 22. The method of embodiment 21, wherein the anti-MIF antibody is selected from the group consisting of RTL100, BAX69, NbE-10, and combinations thereof.

Illustrative embodiment 23. The method of any one of illustrative embodiments 1-22, wherein in step (2), the at least one composition is administered locally/regionally.

Illustrative embodiment 24. The method of any one of illustrative embodiments 1-23, wherein in step (2), the at least one composition is administered systemically.

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

### REFERENCES

Grech N, Dalli T, Mizzi S, Meilak L, Calleja N, Zrinzo A. Rising Incidence of Glioblastoma Multiforme in a Well-Defined Population. Cureus. 2020 May 19; 12(5):e8195. doi: 10.7759/cureus.8195. PMID: 32572354; PMCID: PMC7302718.
Rominiyi, O., Vanderlinden, A., Clenton, S. J. et al. Tumour treating fields therapy for glioblastoma: current advances and future directions. Br J Cancer 124, 697-709 (2021). https://doi.org/10.1038/s41416-020-01136-5.
Novocure® LUNAR: https://novocuretrials.com/cancer-clinical-trials/non-small-cell-lung-cancer-clinical-trial-lunar/(“LUNAR—Non-small Cell Lung Cancer Clinical Trial”); website accessed on 01NOV2023.
Kok T, Wasiel A A, Cool R H, Melgert B N, Poelarends G J, Dekker F J. Small-molecule inhibitors of macrophage migration inhibitory factor (MIF) as an emerging class of therapeutics for immune disorders. Drug Discov Today. 2018 November; 23(11):1910-1918. doi: 10.1016/j.drudis.2018.06.017. Epub 2018 Jun. 21. PMID: 29936245; PMCID: PMC6453109.
Sparkes A, De Baetselier P, Brys L, Cabrito I, Sterckx Y G, Schoonooghe S, Muyldermans S, Raes G, Bucala R, Vanlandschoot P, Van Ginderachter J A, Stijlemans B. Novel half-life extended anti-MIF nanobodies protect against endotoxic shock. FASEB J. 2018 June; 32(6):3411-3422. doi: 10.1096/fj.201701189R. Epub 2018 Jan. 25. PMID: 29401625; PMCID: PMC6137717.
Judy S. Wang, Martin Gutierrez, Drew W. Rasco; et al.; “A phase 1 dose-escalation study to investigate the safety, efficacy, pharmacokinetics, and pharmacodynamic activity of cln-619 (Anti-MICA/B antibody) alone and in combination with pembrolizumab in patients with advanced solid tumors”; Journal of Clinical Oncology 2023 41:16_suppl, 2532.
Pakos-Zebrucka K, Koryga I, Mnich K, Ljujic M, Samali A, Gorman A M. The integrated stress response. EMBO Rep. 2016 October; 17(10):1374-1395. doi: 0.15252/embr.201642195. Epub 2016 Sep. 14. PMID: 27629041; PMCID: PMC5048378.
See, e.g., Isselbacher et al. (1996) Harrison's Principles of Internal Medicine 13 ed., 1814-1882.
Caira M., et al. (2004). Preparation and crystal characterization of a polymorph, a monohydrate, and an ethyl acetate solvate of the antifungal fluconazole. J. Pharmaceut. Sci., 93(3):601-611.
Van Tonder E. C., et al. (2004). Preparation and Physicochemical Characterization of 5 Niclosamide Solvates and 1 Hemisolvate. AAPS Pharm. Sci. Tech., 5(1): Article 12.
Bingham A. L., et al. (2001). Over one hundred solvates of sulfathiazole: solvates and adducts of sulfathiazole. Chem. Commun. 7: 603-604.
Remington's Pharmaceutical Sciences, Mack Publishing Co., Easton, PA, 19th ed. 1995.
Hong, M., Tao, S., Zhang, L.; et al.; RNA sequencing: new technologies and applications in cancer research. J Hematol Oncol 13, 166 (2020). https://doi.org/10.1186/s13045-020-01005-x

We claim:

1. A method of reducing viability of cancer cells and/or of treating a cancer in a subject in need thereof, the method comprising the steps of:
(1) applying an alternating electric field to the cancer cells for a period of time; and
(2) administering at least one composition to the cancer cells, wherein the at least one composition comprises at least one inhibitor of at least one immunomodulatory cytokine selected from the group consisting of macrophage migration inhibitory factor (MIF), MHC class I chain-related polypeptide A (MICA), and/or MHC class I chain-related polypeptide B (MICB).

2. The method of claim 1, wherein at least one of:
the alternating electric field is applied at a frequency in a range of from about 50 kHz to about 1 MHz;
the alternating electric field has a field strength of at least about 1 V/cm in at least a portion of the cancer cells; and
the period of time that the alternating electric field is applied is at least about 50% of a 24 consecutive hour time period.

3. The method of claim 1, wherein the at least one immunomodulatory cytokine inhibitor comprises an anti-MICA/MICB antibody.

4. The method of claim 3, wherein the anti-MICA/MICB antibody comprises one or more of CLN-619 and DM919.

5. The method of claim 1, wherein the at least one immunomodulatory cytokine inhibitor comprises a small molecule inhibitor of MIF.

6. The method of claim 5, wherein the small molecule inhibitor of MIF is selected from the group consisting of ISO-1, 4-IPP, Ibudilast, CPSI 1360, MIF098, MIF-IN-1, MIF-IN-4, MIF-IN-5, MIF-IN-6, R110, and combinations thereof.

7. The method of claim 1, wherein the at least one immunomodulatory cytokine inhibitor comprises an anti-MIF antibody.

8. The method of claim 7, wherein the anti-MIF antibody is selected from the group consisting of RTL100, BAX69, NbE-10, and combinations thereof.

9. The method of claim 1, wherein the cancer is selected from the group consisting of hepatocellular carcinoma, glioblastoma, pleural mesothelioma, differentiated thyroid cancer, advanced renal cell carcinoma, ovarian cancer, pancreatic cancer, lung cancer cell, breast cancer, and combinations thereof.

10. The method of claim 1, further defined as a method of reducing a volume of a tumor and/or preventing an increase of volume of the tumor, wherein the tumor is present in a body of a living subject and includes a plurality of cancer cells.

11. A method for inducing an enhanced/synergistic cytostatic and/or cytotoxic effect on a cancer cell, the method comprising the steps of:
(1) applying an alternating electric field or tumor treating fields (TTFields) to the cancer cell;
(1b) measuring one or more of: an alteration in transcriptional signatures of the cancer cell's cellular metabolism, a modulation in the cancer cell's immune-related cytokines dependent and/or independent of P53, and/or a modulation of the integrated stress response (ISR) either of the cancer cell or a cell in a vicinity of the cancer cell; and
(2) selecting a therapeutic agent using the one or more measurements from step (1b) and contacting the cancer cell with said therapeutic agent; whereby an enhanced effect occurs such that the cytostatic and/or cytotoxic effect on the cancer cell is greater than compared to the cytostatic and/or cytotoxic effect, under the same conditions, that occurs from only the execution of step (1).

12. The method of claim 11, wherein step (1b) comprises:
(1b) measuring a transcriptome and/or a secretome of the cancer cell after or during execution of step (1), and whereby the measuring is further operative to provide a data indicative for selecting a therapeutic agent to exploit an alteration of the cancer cell by the TTFields or alternating electric field.

13. The method of claim 11, further comprising the step of:
(0b) measuring a transcriptome and/or a secretome of the cell before execution of step (1).

14. The method of claim 12, wherein step (1b) is executed after execution of step (1) and one or more changes induced in the transcriptome and/or secretome of the cancer cell after the application of the TTFields are determined via a comparing of a data measured before execution of step (1) (i.e., the data 0b or a pre-treatment data) and a data measured after execution of step (1) (i.e., the data 1b or a post-treatment data).

15. The method of claim 11, wherein the method further comprises the steps of a method of treating a subject in need of a cancer treatment thereof, whereby the following method steps are executed:
(0) obtaining a subject in need of a cancer treatment, subject suspected as having a need for a cancer treatment, or subject in need of a diagnosis for a cancer treatment;
(0b) measuring a transcriptome and/or a secretome of the cancer (or suspected cancer) whereby a pre-treatment data is obtained;
(1) applying an alternating electric field or tumor treating fields (TTFields) to the cancer (or to the suspected cancer area);
(1b) measuring a transcriptome and/or a secretome of the cancer (or suspected cancer area) after or during execution of step (1), whereby a post-treatment data is obtained;
(2) contacting the cancer (or suspected cancer area) with a therapeutic agent, before, after, or during an execution of step (1); wherein the contacting is done by an administration of a therapeutic agent to the subject in need thereof, or an administration of a suitable pharmaceutical formulation, salt, or hydrate thereof; and
whereby the subject is treated more effectively for the cancer (i.e., the extent the cancer is stopped or reversed) by a healthcare provider, substantially as described by the method including the steps of (0b), step (1b), and step (2), than compared to the same treatment, of the same subject, by the same method and the same healthcare provider without any execution of any of the steps of step (0b), step (1b), and step (2).

16. The method of claim 15, wherein the pre-treatment data and/or the post-treatment data is/are compared and/or utilized to determine the therapeutic agent utilized in step (2) and/or a dose or a dose regime of the agent.

17. The method of claim 15, wherein the order of the execution of the steps is changed, the method is repeated or wherein the method is executed as a continuous method.

18. The method of claim 15, wherein the pre-treatment and/or post-treatment data is compared to a previously acquired data from a different subject or to a data contained in a database, wherein the comparison includes a machine learning, e-sending to another location, a comparison by a healthcare provider, and/or a storing for future access.

19. The method of claim 11, whereby the cytostatic and/or cytotoxic effects on the cancer cell are greater than when compared to the same effects of applying TTFields (1) without contacting the cancer cell with the therapeutic agent (2) or vice versa; whereby the cytostatic and/or cytotoxic effects on the cancer cell are greater than when compared to contacting the cancer cell with the therapeutic agent (2) without applying the TTFields (1).
